(19) United States
(12) Patent Application Publication
(10) Pub. No.: US 2021/0155927 A1
(43) Pub. Date: May 27, 2021

(54) METHODS FOR TREATING SICKLE CELL DISEASE

(71) Applicant: The Regents of the University of California, Oakland, CA (US)

(72) Inventors: Mark A. DeWitt, Berkeley, CA (US); David I. Martin, Oakland, CA (US); Wendy Magis, Oakland, CA (US); Jacob E. Corn, Berkeley, CA (US); Mark C. Walters, Alamo, CA (US); Donald B. Kohn, Los Angeles, CA (US); Zulema Romero Garcia, Los Angeles, CA (US)

(21) Appl. No.: 17/047,025

(22) PCT Filed: Apr. 10, 2019
(86) PCT No.: PCT/US19/26806

§ 371 Date: Oct. 12, 2020

Related U.S. Application Data
(60) Provisional application No. 62/657,412, filed on Apr. 13, 2018.

(51) Int. Cl. C12N 15/113; C12N 9/22; C12N 15/10; C12N 5/0789

(52) U.S. Cl. CPC C12N 15/113; C12N 9/22; C12N 15/102; C12N 2510/00; C12N 2310/20; C12N 2310/346; C12N 2310/3181; C12N 5/0647

(57) ABSTRACT

The present disclosure provides a method of modifying a globin gene in the genome of a hematopoietic stem/progenitor cell (HSPC), the method comprising: A) obtaining HSPCs from an individual having a globin gene comprising a sickle cell disease (SCD)-associated single nucleotide polymorphism (SNP) to generate an in vitro population of CD34+ HSPCs and B) contacting the in vitro population with a genome editing composition, as described in further detail below. Also provided is a method of treating sickle cell disease (SCD) in an individual including administering to an individual an in vitro mixed population derived from the method of modifying a globin gene, as well as kits for practicing the same.

### CROSS-REFERENCE

This application claims the benefit of U.S. Provisional Patent Application No. 62/657,412, filed Apr. 13, 2018, which application is incorporated herein by reference in its entirety.

### INCORPORATION BY REFERENCE OF SEQUENCE LISTING PROVIDED AS A TEXT FILE

A Sequence Listing is provided herewith as a text file, “BERK-381PRV_SeqList_ST25.txt” created on Mar. 28, 2018 and having a size of 7,955 KB. The contents of the text file are incorporated by reference herein in their entirety.

### INTRODUCTION

Sickle cell disease is an inherited recessive disease, caused by a single nucleotide polymorphism in β-globin (HBB). The modified hemoglobin causes normally round red blood cells to take on a sticky, sickle-shaped form. Sickle red blood cells clog blood vessels, causing acute pain “crises” and vasculopathy. Additional complications and consequences associated with sickle cell disease include organ damage, organ failure, increased risk of stroke, pulmonary hypertension, acute chest syndrome (ACS), and decreased lifespan. There is no widely available cure for sickle cell disease. Treatments include allogeneic bone marrow transplants, which can be risky and limited by donor availability.

### SUMMARY

The present disclosure provides a method of modifying a globin gene in the genome of a hematopoietic stem/progenitor cell (HSPC), the method comprising: A) obtaining HSPCs from an individual having a globin gene comprising a sickle cell disease (SCD)-associated single-nucleotide polymorphism (SNP) to generate an in vitro population of CD34⁺ HSPCs and B) contacting the in vitro population with a genome editing composition, as described in further detail below. Also provided is a method of treating sickle cell disease (SCD) in an individual including administering to an individual an in vitro mixed population derived from the method of modifying a globin gene, as well as kits for practicing the same.

### BRIEF DESCRIPTION OF THE DRAWINGS

FIG. 1 depicts the co-delivery by electroporation of a Cas9 ribonucleoprotein (RNP) complex and a single-stranded DNA oligonucleotide donor (ssODN) to harvested hematopoietic stem cells (HSCs). A Cas9 RNP is combined with an ssODN to modify the ß-globin gene in HSCs, via electroporation. Purified Cas9 is mixed with purified RNA bearing 3×MS protection. The RNP is mixed with ssODN and HSCs, and the reagents are delivered inside the cells by electroporation. After culture, the cells are analyzed for editing using a next-generation sequencing assay, CFU assay for HSPC multipotent potential, and engraftment in an NBSGW mouse model of engraftment. Edits are maintained in mice, and the edited cells appear healthy and capable of efficient re-population.

FIG. 2 depicts a single-stranded DNA homology directed repair (HDR) donor. The ssDNA HDR donor asymmetrically designed around the cut site in a globin gene can be co-delivered with a Cas9 RNP loaded with a guide RNA by electroporation. (ssDNA Donor: SEQ ID NO: 1123; portion of globin gene with cut site: SEQ ID NO:1124).

FIG. 3 depicts the increase in HbA and HbF in edited SCD CD34⁺ HSPCs compared to non-edited SCD HSPCs.

FIG. 4 depicts the optimization of editing conditions. ER100, DO100, EO100, and CA137 refer to electroporation pulse settings in a Lonza 4D electroporator. 1×MSP refers to the chemical protection of a single guide RNA comprising 2′ O-methyl, thioPACE protection of the terminal 3′ and 5′ nucleotides. 3×MS refers to 2′ O-methyl, phosphoorothioate protection of the three 3′ and three 5′ nucleotides.

FIG. 5 depicts the titration of the RNP complex and an ssDNA donor template for analyzing editing outcomes (NHEJ, HDR) by next-generation sequencing in CD34⁺ HSPCs.

FIG. 6A-6F provides amino acid sequences of Streptococcus pyogenes Cas9 (FIG. 6A) and variants of Streptococcus pyogenes Cas9 (FIG. 6B-6F).

FIG. 7 provides an amino acid sequence of Staphylococcus aureus Cas9.

FIG. 8A-8C provide amino acid sequences of Francisella tularensis Cpf1 (FIG. 8A), Acidaminococcus sp. BV3L6 Cpf1 (FIG. 8B), and a variant Cpf1 (FIG. 8C).

FIG. 9 depicts the percent of viable unedited and edited SCD HSPCs that were mobilized by plerixafor, as well as the percent of corrected SCD alleles in HSPCs mobilized by plerixaflor. 10⁸ HSPCs were collected after mobilization by plerixafor.

FIG. 10 shows in vitro phenotyping of HSPCs by HPLC. FIG. 10 depicts a chromatogram obtained from edited Plerixafor-mobilized SCD CD34⁺ HSPCs after correction with an ssDNA donor. The HSPCs included 22% HbS, 40% HbA, 37% HbF, and 78% non-sickle hemoglobin.

FIG. 11 shows in vitro phenotyping by RNAseq. The analyzed sample included >50% non-sickle HBB.

FIG. 12 depicts a schematic of the protocol for in vitro editing of SCD-associated SNPs in HSPCs and injection of the edited HSPCs into mice.

FIG. 13 depicts the percent of corrected SCD alleles after four months of engraftment in NBSGW mice. After engraftment, analysis showed >90% elimination of the SCD allele as well as an average correction of 22% in marrow and 20% in progenitors in marrow. The engraftment average was 45%.

FIG. 14 depicts cutting of on- and off-target sites by high—fidelity Cas9 variants, as well as viability of the high-fidelity Cas9 variants after electroporation. IDT mutant 1 reduced off-target cutting by 20 times.

FIG. 15 depicts a genomic region of the human beta-globin gene and the location of the SCD-associated SNP. The SCD-associated SNP (A to T mutation) is located at position 170 (bold). The PAM sequence (positions 182-184; underline and bold) and G10 guide RNA binding site (positions 185-204; underline) are also depicted.

FIG. 16 depicts the sequence of ssDNA donor CJ6A. The ssDNA donor CJ6A may be used to correct the SCD-associated SNP to the wild-type SNP.

FIG. 17 depicts the sequence of an embodiment of a guide RNA (G10) and a guide RNA targeting segment. “*” denotes 2′O-methyl phosphorothioate protection.

FIG. 18 depicts clinical-scale electroporation of harvested CD34⁺ HSPCs with RNP/ss donor DNA template. Following electroporation, the cells are frozen, then thawed prior to use.

### DEFINITIONS

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a ssRNA target nucleic acid base pairs with a DNA PAM-containing oligonucleotide (also referred to herein as a “PAMmer”), when a DNA target nucleic acid base pairs with a guide RNA, etc.): guanine (G) can also base pair with uracil (U). For example, G/U base-pairing is partially responsible for the degeneracy (i.e., redundancy) of the genetic code in the context of tRNA anti-codon base-pairing with codons in mRNA. Thus, in the context of this disclosure, a guanine (G) (e.g., of a protein-binding segment (dsRNA duplex) of a guide RNA molecule; of a target nucleic acid base pairing with a guide RNA and/or a PAM-containing oligonucleotide, etc.) is considered complementary to both a uracil (U) and to an adenine (A). For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or fewer, 30 or fewer, 25 or fewer, 22 or fewer, 20 or fewer, or 18 or fewer nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). The temperature and wash solution salt concentration may be adjusted as necessary according to factors such as length of the region of complementation and the degree of complementation.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Exemplary methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

A “target nucleic acid” or “target segment” as used herein is a polynucleotide (e.g., RNA, DNA) that includes a “target site” or “target sequence.” The terms “target site” or “target sequence” are used interchangeably herein to refer to a nucleic acid sequence present in a target nucleic acid to which a targeting segment of a guide RNA will bind, provided sufficient conditions for binding exist; and/or to which a region (segment) of a PAM-containing oligonucleotide (e.g., a specificity segment and/or an orientation segment) will bind. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide RNA is referred to as the “complementary strand”; while the strand of the target nucleic acid that is complementary to the “complementary strand” (and is therefore not complementary to the guide RNA) is referred to as the “noncomplementary strand” or “non-complementary strand”. In cases where the target nucleic acid is a single stranded target nucleic acid (e.g., single stranded DNA (ssDNA), single stranded RNA (ssRNA)), the guide RNA is complementary to and hybridizes with single stranded target nucleic acid.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. In certain embodiments, a complex comprising a guide RNA and a Class 2 CRISPR effector protein is used for targeted cleavage of a single stranded target nucleic acid (e.g., ssRNA, ssDNA).

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

A nucleic acid molecule that binds to the Class 2 CRISPR effector protein and targets the protein to a specific location within the target nucleic acid is referred to herein as a “guide RNA”. A guide RNA comprises two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. For example, in some cases the guide RNA is one nucleic acid molecule (e.g., one RNA molecule) and the protein-binding segment therefore comprises a region of that one molecule. In other cases, the protein-binding segment (described below) of a guide RNA includes regions of two separate molecules that are hybridized along a region of complementarity (forming a dsRNA duplex). The definition of “segment,” unless otherwise specifically defined in a particular context, is not limited to a specific number of total base pairs, is not limited to any particular number of base pairs from a given nucleic acid molecule, is not limited to a particular number of separate molecules within a complex, and may include regions of nucleic acid molecules that are of any total length and may or may not include regions with complementarity to other molecules.

In some embodiments, a subject nucleic acid (e.g., a guide RNA, a nucleic acid comprising a nucleotide sequence encoding a guide RNA; a nucleic acid encoding a Class 2 CRIPSR effector protein; a PAM-containing oligonucleotide, etc.) comprises a modification or sequence (e.g., an additional segment at the 5′ and/or 3′ end) that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a ribozyme sequence (e.g. to allow for self-cleavage and release of a mature molecule in a regulated fashion); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the nucleic acid to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA and/or RNA, including transcriptional activators, transcriptional repressors, DNA methyltransferases, DNA demethylases, histone acetyltransferases, histone deacetylases, and the like); and combinations thereof.

A guide RNA and a Class 2 CRISPR effector protein form a complex (i.e., bind via non-covalent interactions). The guide RNA provides target specificity to the complex by comprising a nucleotide sequence that is complementary to a sequence of a target nucleic acid. The protein of the complex provides the site-specific activity. In other words, the protein is guided to a target nucleic acid sequence (e.g. a target sequence in a chromosomal nucleic acid; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; etc.) by virtue of its association with the protein-binding segment of the guide RNA.

In some embodiments, a guide RNA comprises two separate nucleic acid molecules: an “activator” and a “targeter” (see below) and is referred to herein as a “dual guide RNA”, a “double-molecule guide RNA”, a “dual guide RNA”, a “two-molecule guide RNA”, or simply “dgRNA.” In some embodiments, the guide RNA has an activator and a targeter (as are present in a dual guide RNA), where the activator and targeter are covalently linked to one another (e.g., via intervening nucleotides) and is referred to herein as a “single guide RNA”, a “single-molecule guide RNA,” or a “one-molecule guide RNA.” The term “guide RNA” is inclusive, referring to both dual guide RNAs (dgRNAs) and to single guide RNAs (sgRNAs). In some cases, a guide RNA is a DNA/RNA hybrid molecule.

As used herein, “globin gene” refers to a gene that encodes a polypeptide of a hemoglobin molecule. The globins are a superfamily of heme-containing globular proteins which are involved in binding and transporting oxygen. There are two main clusters of globin genes in humans: the alpha globin cluster on chromosome 16 and the beta globin cluster on chromosome 11. In humans, the normal hemoglobin molecule consists of four polypeptide chains, the α-, β-, δ- and γ-globin chains, which are encoded by the α-, β-, δ- and γ-globin genes, respectively. Further, in the human adult, there are three different hemoglobin types made up of different combinations of these globin chains: Hemoglobin A (HbA), Hemoglobin A2 (I-IhA2), and Hemoglobin F (HhF). Hemoglobin A (HbA), the predominant type of hemoglobin in adults is made of 2 α-chains and 2 β-chains.

As used herein, “sickle cell disease” (SCD) refers to a group of genetic disorders characterized by the predominance of hemoglobin S (HbS). These disorders include, for example, sickle cell anemia, the sickle beta thalassemia syndromes, and hemoglobinopathies in which HbS is in association with another abnormal hemoglobin. SCD is a severe hemoglobinopathy that produces multisystem complications due to the expression of abnormal sickle hemoglobin (HbS). The most common type of SCD is sickle cell anemia (SCA) (also referred to as HbSS or SS disease or hemoglobin S) in which there is homozygosity for the mutation that causes HbS. The more rare types of SCD in which there is heterozygosity (one copy of the mutation that causes HbS and one copy for another abnormal hemoglobin allele) for the mutation include sickle-hemoglobin C (HbSC), sickle β⁺ thalassemia (HbS/β⁺) and sickle) β⁰ thalassemia)(HbS/β⁰.

As used herein, “stem cell mobilization agent” refers to any agent that facilitates or enhances the mobilization of hematopoietic stem/progenitor cells (HSPCs), e.g., from the bone marrow (BM) to the peripheral blood (PB). The mobilized HSPCs may be preserved, frozen, and stored until the time of transplant or reinfusion. As used herein, the term “hematopoietic stem/progenitor cells” refers to a heterogeneous population of cells including hematopoietic progenitor cells and hematopoietic stem cells. It is also contemplated herein that hematopoietic stem cells and/or hematopoietic progenitor cells are isolated and expanded ex vivo prior to transplantation.

As used herein, the term “hematopoietic progenitor cells” encompasses pluripotent cells capable of differentiating into several cell types of the hematopoietic system, including, but not limited to, granulocytes, monocytes, erythrocytes, megakaryocytes, B-cells and T-cells. Hematopoietic progenitor cells are committed to the hematopoietic cell lineage and generally do not self-renew. The term “hematopoietic progenitor cells” encompasses short term hematopoietic stem cells (ST-HSCs), multi-potent progenitor cells (MPPs), common myeloid progenitor cells (CMPs), granulocyte-monocyte progenitor cells (GMPs), and megakaryocyte-erythrocyte progenitor cells (MEPs). The term “hematopoietic progenitor cells” does not encompass hematopoietic stem cells capable of self-renewal (herein referred to as “hematopoietic stem cells”). The presence of hematopoietic progenitor cells can be determined functionally as colony forming unit cells (CFU-Cs) in complete methylcellulose assays, or phenotypically through the detection of cell surface markers using assays known to those of skill in the art.

As used herein, the term “hematopoietic stem cell (HSC)” refers to a cell with multi-lineage hematopoietic differentiation potential and sustained self-renewal activity. “Self renewal” refers to the ability of a cell to divide and generate at least one daughter cell with the identical (e.g., self-renewing) characteristics of the parent cell. The second daughter cell may commit to a particular differentiation pathway. For example, a self-renewing hematopoietic stem cell divides and forms one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Hematopoietic stem cells have the ability to regenerate long term multi-lineage hematopoiesis (e.g., “long-term engraftment”) in individuals receiving a bone marrow or cord blood transplant. The hematopoietic stem cells used may be derived from any one or more of the following sources: fetal tissues, cord blood, bone marrow, peripheral blood, mobilized peripheral blood, a stem cell line, or may be derived ex vivo from other cells, such as embryonic stem cells, induced pluripotent stem cells (iPS cells) or adult pluripotent cells. The cells from the above listed sources may be expanded ex vivo using any method acceptable to those skilled in the art prior to use in the transplantation procedure. For example, cells may be sorted, fractionated, treated to remove malignant cells, or otherwise manipulated to treat the patient using any procedure acceptable to those skilled in the art of preparing cells for transplantation. If the cells used are derived from an immortalized stem cell line, further advantages would be realized in the ease of obtaining and preparation of cells in adequate quantities.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a hematopoietic stem cell” includes a plurality of such hematopoietic stem cells and reference to “the class 2 CRISPR/Cas effector polypeptide” includes reference to one or more class 2 CRISPR/Cas effector polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a method of modifying a globin gene in the genome of a hematopoietic stem/progenitor cell (HSPC), the method comprising: A) obtaining HSPCs from an individual having a globin gene comprising a sickle cell disease (SCD)-associated single-nucleotide polymorphism (SNP) to generate an in vitro population of CD34⁺ HSPCs and B) contacting the in vitro population with a genome editing composition, as described in further detail below. Also provided is a method of treating sickle cell disease (SCD) in an individual including administering to an individual an in vitro mixed population derived from the method of modifying a globin gene, as well as kits for practicing the same.

### Methods of Modifying a Globin Gene

The present disclosure provides a method of modifying a globin gene in the genome of a hematopoietic stem/progenitor cell (HSPC). The method may include the steps of: A) obtaining HSPCs from an individual having a globin gene comprising a sickle cell disease (SCD)-associated single-nucleotide polymorphism (SNP), wherein said obtaining comprises: a) administering to the individual an amount of a stem cell mobilization agent effective to mobilize CD34⁺ HSPCs; and b) collecting the mobilized CD34⁺ HSPCs from the individual, thereby generating an in vitro population of CD34⁺ HSPCs; B) contacting the in vitro population of CD34⁺ HSPCs with a genome editing composition comprising: a) a ribonucleoprotein (RNP) complex comprising: i) a class 2 CRISPR/Cas effector polypeptide, or a nucleic acid comprising a nucleotide sequence encoding the class 2 CRISPR/Cas effector polypeptide; and ii) a guide RNA; and b) a donor DNA template comprising a nucleotide sequence that provides for correction of the SCD-associated SNP in the globin gene, thereby generating an in vitro mixed population, wherein at least 2% %, e.g., at least 5%, at least 10%, at least 15%, at least 20%, at least 25%, at least 30%, at least 35%, at least 40%, at least 45%, at least 50%, or more than 50%, of the SCD-associated SNPs have been corrected in the in vitro mixed population. As used herein, the terms “corrected” and “edited” may be used interchangeably. As used herein, the terms “globin gene” may be used interchangeably with “globin allele.” In some cases, a globin allele comprises a sickle cell mutation, referred to herein as “a sickle cell allele” or “SCD allele.” A “corrected SCD allele” may refer to a β-globin allele in which an SCD-associated SNP has been corrected, such that the SCD-associated SNP is no longer present in the allele; i.e., such that the allele encodes a β-globlin that does not include a SCD mutation. A “corrected SCD-associated SNP” may be used interchangeably with “a corrected β-globin allele.” A globin allele comprising a corrected SCD allele may refer to a globin allele comprising a corrected SCD-associated SNP or a globin allele having no SCD-associated SNPs.

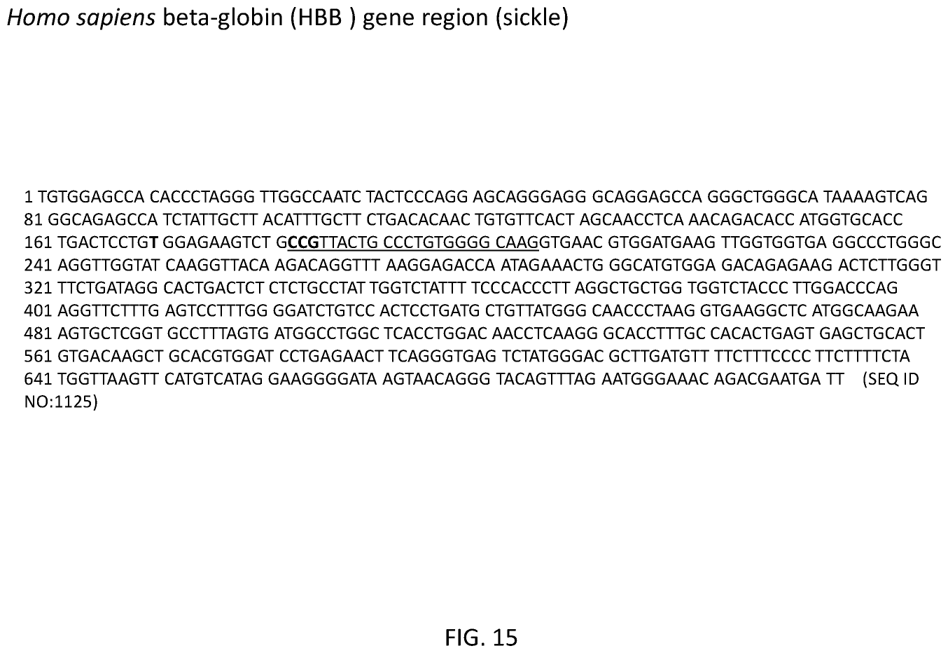

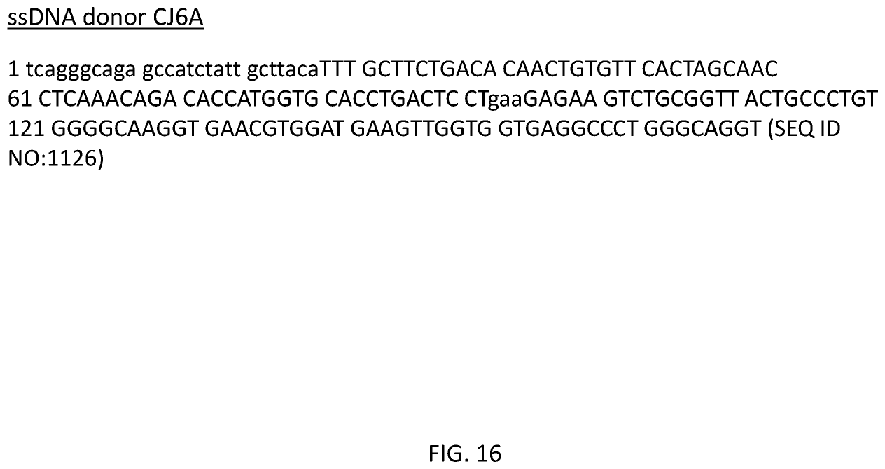

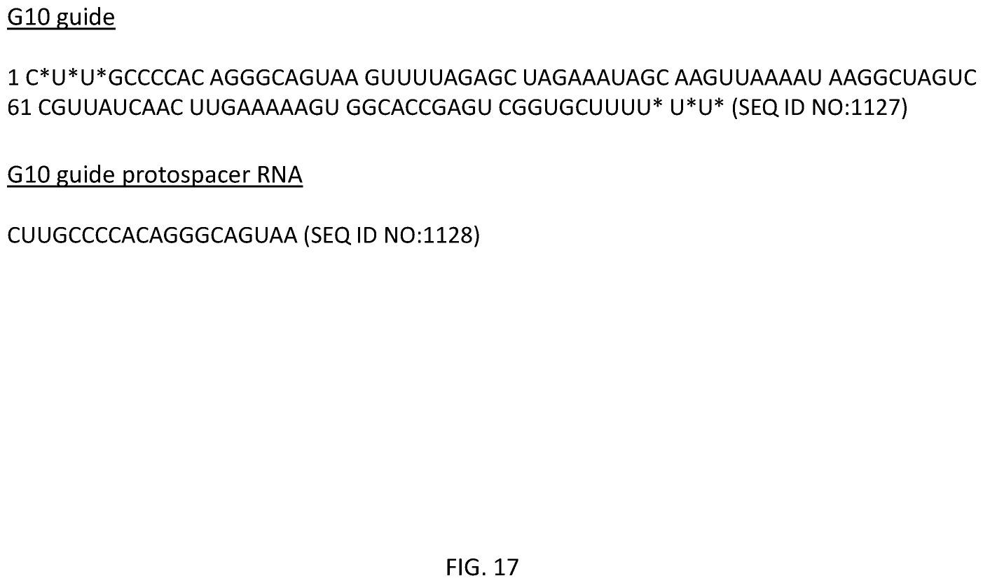

Aspects of the methods include obtaining HSPCs from an individual having a globin gene comprising a SCD-associated SNP. Various SCD-associated SNPs may be suitable for editing in the subject methods. In some cases, the SCD-associated SNP is an A-to-T substitution at position 170 of the nucleotide sequence depicted in FIG. 15. In some cases, the HSPCs are obtained from an individual who is homozygous for a SCD-associated SNP. In some cases, the HSPCs are obtained form an individual who is heterozygous for a SCD-associated SNP.

Aspects of the methods include administering an amount of stem cell mobilization agent. The stem cell mobilization agent may be used to obtain a sample of CD34⁺ HSPCs from an individual. In some cases, the stem cell mobilization agent is a small molecule. In some instances, the stem cell mobilization agent is a cytokine. Suitable stem cell mobilization agents include, but are not limited to, AMD3465, NIBR 1816, TG-0054, G-CSF, GM-CSF, SDF-1, and SCF. In some cases, the stem cell mobilization agent is plerixafor, as described in detail in U.S. Pat. No. 7,897,590; U.S. Pat. No. RE42,152; and U.S. Pat. No. 6,987,102. Plerixafor is a macrocyclic compound and a hicvclam derivative having the structure:

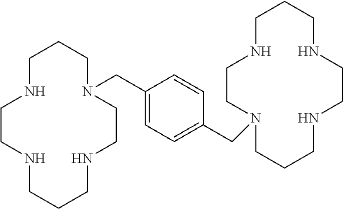 [SMILES: c1cc(CN2CCCNCCNCCCNCC2)ccc1CN1CCCNCCNCCCNCC1]

### Structure 1

### 1,4-Bis 1,4,8,11-tetraazacyclotetradecan-1-yl)methyl)benzene

An effective amount of the stem cell mobilization agent can vary and may depend on the stem cell mobilization agent. In some cases, an effective amount is the amount effective to mobilize from about 10⁵ CD34⁺ HSPCs to about 10⁸ CD34⁺ HSPCs. Where the stem cell mobilization agent is plerixafor, an effective amount to mobilize the requisite amount of CD34⁺ HSPCs can range from about 200 μg to about 300 μg (e.g., from about 200 μg to about 220 μg, from about 220 μg to about 240 μg, from about 240 μg to about 250 μg, or from about 250 to about 300 μg. In some cases, 240 μg plerixafor is administered to an individual by a subcutaneous injection 5-10 hours before HSPC harvesting by aphersis. Subjects can also undergo an RBC exchange transfusion completed before the harvesting apheresis) procedure, to reduce the circulating HbS fraction to 30% and thereby reduce the risk of a vaso-occlusive complication during the plerixafor mobilization and apheresis procedure. In some cases, the target yield for this procedure is 10×10⁶ CD34⁺ cells/kg recipient weight. In some cases, the aphersis procedure is performed for up to 2 consecutive days. In some cases, an effective amount is the amount effective to mobilize from about 10⁵ HSPCs to 10⁸ FISPCs, such as, e.g., from 10⁵ to 10⁶ HSPCs, from 10⁶ to 10⁷ HSPCs, from 10⁷ to 10⁸ HSPCs, or more than 10⁸ HSPCs. The mobilized stem cells may be collected, thereby generating an in vitro population of CD34⁺ HSPCs. The in vitro population of HSPCs can include from 10⁵ to 10⁸ cells such as, e.g., from 10⁵ to 10⁶ cells, from 10⁶ to 10⁷ cells, from 10⁷ to 10⁸ cells, or more than 10⁸ cells. The in vitro population of CD34⁺ HSPCs may be cultured for a period of time before the population is contacted with a genome editing composition, as described below. In some cases, the in vitro population of unedited HSPCs may be cultured for 1 hour (hr) to 80 hours (hrs) such as, e.g., for 1 hr to 72 hrs, for 1 hr to 48 hrs, for 1 hr to 24 hrs, for 1 hr to 10 hrs, for 1 hr to 5 hrs, or for 1 hr to 2 hrs. The culture media may include the following: growth factors, cytokines, adhesion mediators, minerals, among other factors. Additional culture parameters that may be suitable are described in Frisch, B. J., & Calvi, L. M. (2014). Hematopoietic Stem Cell Cultures and Assays. Methods in Molecular Biology (Clifton, N.J.), 1130, 315-324; Potter, H., & Heller, R. (2003). Transfection by Electroporation. Current Protocols in Molecular Biology/Edited by Frederick M. Ausubel et al., CHAPTER, Unit-9.3.

The in vitro population of CD34⁺ HSPCs may be isolated or purified from a sample by any known method. In certain embodiments, the HSPCs may be magnetically labeled and separated from a sample with use of a magnetic field generated by a magnetic field source, e.g., a permanent magnet or an electromagnet. The HSPCs may be labeled with magnetic particles such as, e.g., ferromagnetic, superparamagnetic or paramagnetic solid phases such as colloidal particles, microspheres, nanoparticles, or beads. The particles may be used in suspension or in a lyophilized state. In certain embodiments, the magnetically labeled cells are separated from a sample in a magnetic activated cell separation (MACS®) system. The technique of magnetic activated cell sorting can involve coupling a cell surface with magnetic particles the size of cellular macromolecules. The cells may be passed through a magnetizable matrix in a strong magnetic field. Labeled cells may stick to the matrix and can be separated form unlabeled cells, which flow through. The magnetic labeled cells can be eluted when the column is demagnetized by removal from the magnetic field. In some instances, the system includes a magnetic separator, i.e., an apparatus containing one or magnets, e.g., one or more permanent magnets, and configured to hold one or more magnetic separation columns. The separation columns for use with the magnetic separator include columns that may be filled with a paramagnetic material, e.g., iron spheres, to amplify the magnetic field of the magnetic separator. The magnetic field retains magnetically labeled cells that pass through the column placed in a separator. In some instances, the separator may be a MACS separator, e.g., CliniMACS® separator, MiniMACS™ separator, MidiMACS™ separator, etc. In some instances, the column may be a MACS column, e.g., MACS® MS column, MACS® LS Column, etc.

Aspects of the methods include contacting the in vitro population of CD34⁺ HSPCs with a genome editing composition. The number of HSPCs in the in vitro population for contacting with a gene editing composition may range from 10⁵ to 5×10⁹ cells such as, e.g., from 10⁵ to 10⁶ cells from 10⁶ to 10⁷ cells, from 10⁷ to 10⁸ cells, from 10⁸ cells to 5×10⁸ cells, from 5×10⁸ cells to 10⁹ cells, from 10⁹ cells to 2×10⁹ cells, or from 2×10⁹ cells to 5×10⁹ cells. The genome editing composition may include an RNP complex comprising a class 2 CRISPR/Cas effector polypeptide or a nucleic acid comprising a nucleotide sequence encoding the class 2 CRISPR/Cas effector polypeptide. The RNP complex may further comprise a guide RNA or a nucleic acid comprising a nucleotide sequence encoding the guide RNA. The genome editing composition may further include a donor DNA template (e.g., a single-stranded donor DNA template, as described below) comprising a nucleotide sequence that provides for correction of the SCD-associated SNP in the globin gene. The contacting may include combining, incubating, or mixing the genome editing composition with the in vitro population of CD34⁺ HSPCs. In some cases, the genome editing composition may be introduced into a cell, e.g., an HSPC. The genome editing composition may be introduced into a cell by any known method in the art such as, e.g., electroporation. A Class 2 CRISPR effector protein or nucleic acid encoding the Class 2 CRISPR effector protein may be introduced inside a cell. A guide RNA or nucleic acid encoding the guide RNA may be introduced into a cell. In some cases, the guide RNA has the nucleotide targeting segment 5′-CUUGCCCCACAGGGCAGUAA-3′ (SEQ ID NO: 1128). A donor DNA template may be introduced into a cell. The donor DNA template is in some cases single-stranded DNA. In some cases, the donor DNA template includes the nucleotide sequence

(SEQ ID NO: 1126)
5′-tcagggcagagccatctattgcttacaTTTGCTTCTGACACAACTGTG
TTCACTAGCAACCTCAAACAGACACCATGGTGCACCTGACTCCTgaaGAGA
AGTCTGCGGTTACTGCCCTGTGGGGCAAGGTGAACGTGGATGAAGTTGGTG
GTGAGGCCCTGGGCAGGT-3′.

The in vitro HSPC population may be contacted with any suitable amounts of the genome editing composition or components of the genome editing composition. In some cases, the amount of the RNP complex ranges from 10 pmol to 150 pmol per 10⁵ cells such as, e.g., from 50 pmol to 125 pmol, from 55 pmol to 120 pmol, from 60 pmol to 115 pmol, from 65 pmol to 110 pmol, from 70 pmol to 100 pmol, or from 75 pmol to 90 pmol per 10⁵ cells. In some cases, the amount of the ssDNA donor template ranges from 10 pmol to 150 pmol per 10⁵ cells such as, e.g., from 60 pmol to 140 pmol, from 70 pmol to 130 pmol, from 80 pmol to 120 pmol, from 90 pmol to 110 pmol, or 100 pmol to 105 pmol per 10⁵ cells. In some cases, e.g., for electroporation, the RNP complex, the ssDNA donor template, and the in vitro HSPCs are in a volume of from 1 μL to 30 μL; for example, the volume can range from 1 μL to 25 μL, from 5 μL to 20 μL, or from 10 μL to 20 μL. Volumes for clinical-scale gene-editing range from about 1 mL to about 100 mL (e.g., from about 1 mL to about 2 mL, from about 2 mL to about 5 mL, from about 5 mL to about 10 mL, from about 10 mL to about 25 mL, from about 25 mL to about 50 mL, from about 60 mL to about 75 mL, or from about 75 mL to about 100 mL). For example, a gene-editing composition suitable for use in a clinical setting with from about 10⁸ cells to about 10⁹ cells comprises e.g., from about 2 μM to about 5 μM ssDNA donor, from about 2 μM to about 5 μM Cas9, and from about 2 μM to about 5 μM RNA in from 1 mL to about 100 mL (e.g., from about 1 mL to about 2 mL, from about 2 mL to about 5 mL, from about 5 mL to about 10 mL, from about 10 mL to about 25 mL, from about 25 mL to about 50 mL, from about 60 mL to about 75 mL, or from about 75 mL to about 100 mL) of solution.

The contacting may occur under conditions suitable for a reaction to occur, e.g., for enzymatic cleavage to occur, for correction of the SCD-associated SNP to occur, for generation of the in vitro mixed population to occur. In some cases, the contacting occurs after culturing the in vitro population of unedited HSPCs. In some cases, the contacting to produce an in vitro mixed population occurs for a period of time that is less than 1 hour; for example, the contacting may occur for a period of time that is less than 45 min, less than 30 min, less than 20 min, less than 10 min, less than 5 min, or less than 1 min. In some instances, the contacting occurs at room temperature. A variety of other reagents may be included in the generation of the in vitro mixed population. These include reagents such as nuclease inhibitors, protease inhibitors, solubilizing agents, and the like. Reagents that improve the efficiency of the production of the in vitro mixed population include, but are not limited to, salts, peptides that bind Cas9, peptides that bind the pile RNA, nucleic acids that bind Cas9, nucleic acids that bind the guide RNA, small molecules that bind Cas9, or small molecules that bind the guide RNA, etc. The mixture of components can be added during an assay in any order that provides for the in vitro mixed population. In some cases, the in vitro population of unedited HSPCs is contacted with a gene editing composition and subjected to electroporation. In some cases, a mixture for use in electroporation, i.e., “an electroporation mixture,” includes any suitable electroporation buffer, Cas9 buffer (150 mM KCl, 50 mM HEPES pH 7.5, 10-50% glyercol), and gene editing components (e.g., Cas9 protein, a guide RNA, and an ssDNA HDR donor). In some cases, the volume of the electroporation mixture ranges from 20 μL to 100 μL; for example, the volume of the electroporation mixture can range from 20 μL to 50 μL, from 50 μL to 75 μL, or from 75 μL to 100 μL. In some cases, the volume of the electroporation mixture ranges from 1 mL to about 100 mL (e.g., from about 1 mL to about 2 mL, from about 2 mL to about 5 mL, from about 5 mL to about 10 mL, from about 10 mL to about 25 mL, from about 25 mL to about 50 mL, from about 60 mL to about 75 mL, or from about 75 mL to about 100 mL). Electroporation protocols for introducing gene editing components in cells are well known in the art. See, e.g., Potter, H., & Heller, R. (2003). Transfection by Electroporation. Current Protocols in Molecular Biology/Edited by Frederick M. Ausubel . . . [et al.], CHAPTER, Unit-9.3; and Jacobi, A. M., Rettig, G. R., Turk, R., Collingwood, M. A., Zeiner, S. A., Quadros, R. M., . . . Behlke, M. A. (2017). Simplified CRISPR tools for efficient genome editing and streamlined protocols for their delivery into mammalian cells and mouse zygotes. Methods, 121-122, 16-28. doi:10.1016/j.ymeth.2017.03.021.

After electroporation has occurred, the in vitro mixed HSPC population may be cultured in vitro for a period of time. The in vitro mixed HSPC population may be cultured for a period of time ranging from 0 days to 7 days such as, e.g., from 0 days to 6 days, from 0 days to 5 days, from 0 days to 4 days, from 0 days to 3 days, from 0 hours (hr) to 48 hrs, from 0 hr to 24 hrs, from 0 hr to 10 hrs, from 0 hr to 5 hrs, or from 0 hr to 2 hrs. The in vitro mixed HSPC population may be cultured in the presence of any suitable factors to promote the growth and expansion of the in vitro mixed population, e.g., HSPCs in the in vitro mixed population, including, but not limited to, the following: growth factors, adhesion mediators, minerals, cytokines (e.g., stem cell factor (SCF), Flt-3 ligand, thrombopoietin (TPO)), IL-3, IL-6, G-CSF, and animal-free stem cell culture media (e.g., SFEM II from StemCell Technologies; X-VIVO™ 15 (chemically defined, serum-free hematopoietic cell culture medium) from Lonza; and the like) among other factors. Additional culture parameters that may be suitable are described in Frisch, B. J., & Calvi, L. M. (2014). Hematopoietic Stem Cell Cultures and Assays. Methods in Molecular Biology (Clifton, N.J.), 1130, 315-324; Potter, H., & Heller, R. (2003). Transfection by Electroporation. Current Protocols in Molecular Biology/Edited by Frederick M. Ausubel . . . [et Al.], CHAPTER, Unit-9.3; and Jacobi, A. M., Rettig, G. R., Turk, R., Collingwood, M. A., Zeiner, S. A., Quadros, R. M., . . . Behlke, M. A. (2017). Simplified CRISPR tools for efficient genome editing and streamlined protocols for their delivery into mammalian cells and mouse zygotes. Methods, 121-122, 16-28. doi:10.1016/j.ymeth.2017.03.021.

The contacting may generate an in vitro mixed population. As used herein, the term “in vitro mixed population” refers to an in vitro population of genome editing composition-contacted CD34⁺ HSPCs. The term “in vitro mixed population” may be used interchangeably with “in vitro mixed HSPC population.” The cells of the in vitro mixed population may include viable HSCs capable of engraftment and long-term self-renewal. The in vitro mixed population may include three populations of cells: 1) a population of cells that have two non-corrected β-globin alleles with SCD-associated SNPs; 2) a population of cells that have only one β-globin allele with an SCD-associated SNP that has been corrected; and 3) a population of cells that have two β-globin alleles with SCD-associated SNPs that have been corrected. In some cases, the population of cells having two non-corrected β-globin alleles includes cells where one or more β-globin alleles have been knocked out. The knockout of one or more β-globin alleles may be due to non-homologous end joining (NHEJ) where small insertions or deletions (indels) are inserted at the site of cleavage, where the indels cause functional disruption through introduction of non-specific mutations at the cleavage location. The in vitro mixed population of cells may include the following percentages of the three populations of cells as described above: (90% of the total cells have two non-corrected β-globin alleles, 5% of the total cells have one corrected allele, 5% of the total cells have two corrected alleles); (80% of the total cells have two non-corrected β-globin alleles, 10% of the total cells have one corrected allele, 10% of the total cells have two corrected alleles); (70% of the total cells have two non-corrected β-globin alleles, 15% of the total cells have one corrected allele, 15% of the total cells have two corrected alleles); (60% of the total cells have two non-corrected β-globin alleles, 20% of the total cells have one corrected allele, 20% of the total cells have two corrected alleles); (50% of the total cells have two non-corrected β-globin alleles, 25% of the total cells have one corrected allele, 25% of the total cells have two corrected alleles); (40% of the total cells have two non-corrected β-globin alleles, 30% of the total cells have one corrected allele, 30% of the total cells have two corrected alleles); (30% of the total cells have two non-corrected β-globin alleles, 35% of the total cells have one corrected allele, 35% of the total cells have two corrected alleles); (20% of the total cells have two non-corrected β-globin alleles, 40% of the total cells have one corrected allele, 40% of the total cells have two corrected alleles); (10% of the total cells have two non-corrected β-globin alleles, 45% of the total cells have one corrected allele, 45% of the total cells have two corrected alleles); (0% of the total cells have two non-corrected β-globin alleles, 50% of the total cells have one corrected allele, 50% of the total cells have two corrected alleles). In certain embodiments, 2% to 95% of cells of the in vitro mixed population comprise two non-corrected SCD-associated SNPs after a period of time such as, e.g., 2% to 90% of cells, 2% to 80% of cells, 2% to 70% of cells, 2% to 60% of cells, 2% to 50% of cells, 2% to 40% of cells, 2% to 30% of cells, or 2% to 20% of cells. In certain embodiments, 2% to 95% of cells of the in vitro mixed population comprise only one corrected SCD-associated SNP after a period of time such as, e.g., 2% to 90% of cells, 2% to 80% of cells, 2% to 70% of cells, 2% to 60% of cells, 2% to 50% of cells, 2% to 40% of cells, 2% to 30% of cells, or 2% to 20% of cells. In certain embodiments, 2% to 95% of cells of the in vitro mixed population comprise two corrected SCD-associated SNPs after a period of time such as, e.g., 2% to 90% of cells, 2% to 80% of cells, 2% to 70% of cells, 2% to 60% of cells, 2% to 50% of cells, 2% to 40% of cells, 2% to 30% of cells, or 2% to 20% of cells. In certain embodiments, 2% to 95% of cells from the in vitro mixed population comprise at least one corrected SCD-associated SNP after a period of time such as, e.g., 2% to 90% of cells, 2% to 80% of cells, 2% to 70% of cells, 2% to 60% of cells, 2% to 50% of cells, 2% to 40% of cells, 2% to 30% of cells, or 2% to 20% of cells. The period of time may be a period of time after contacting of the in vitro population with the genome editing composition and may range from 0 days to 7 days such as, e.g., from 0 days to 6 days, from 0 days to 5 days, from 0 days to 4 days, from 0 days to 3 days, from 0 hours (hr) to 48 hrs, from 0 hr to 24 hrs, from 0 hr to 10 hrs, from 0 hr to 5 hrs, or from 0 hr to 2 hrs. In some cases, the percentage of the β-globin alleles with SCD-associated SNPs that have been corrected in the in vitro mixed population is at least 2%; for example at least 2%, at least 5%, at least 10%, at least 15%, at least 20%, at least 25%, at least 30%, at least 35%, at least 40%, at least 45%, at least 50%, or more than 50% of the β-globin alleles with SCD-associated SNPs have been corrected. In some cases, at least 2% of the β-globin alleles with SCD-associated SNPs have been corrected; for example at least 2%, at least 5%, at least 10%, at least 15%, at least 20%, at least 25%, at least 30%, at least 35%, at least 40%, at least 45%, at least 50%, or more than 50%, of the β-globin alleles in the in vitro mixed population have a corrected SCD-associated SNP. In some cases, 2% to 60% of the SCD-associated SNPs in the in vitro mixed population have been corrected; for example, 2% to 50%, 2% to 40%, 2% to 30%, 2% to 25%, 2% to 20%, or 2% to 10% of the SCD-associated SNPs in the in vitro mixed population have been corrected. In some cases, 50% of the SCD-associated SNPs in the in vitro mixed population have been corrected; for example, 45% of the SCD-associated SNPs, 35% of the SCD-associated SNPs, 25% of the SCD-associated SNPs, or 15% of the SCD-associated SNPS in the in vitro mixed population have been corrected. In some cases, the in vitro mixed population includes a population of HSCs with at least one β-globin allele with an SCD-associated SNP that has been corrected. A β-globin allele with a “corrected SCD-associated SNP” encodes a polypeptide subunit for forming HbA (and not HbS). The in vitro mixed population may be cultured for a period of time before the population is administered to an individual, as described below. In some cases, the in vitro mixed HSPC population (comprising edited HSPCs) may be cultured for 0 days to 7 days such as, e.g., from 0 days to 6 days, from 0 days to 5 days, from 0 days to 4 days, from 0 days to 3 days, from 0 hours (hr) to 48 hrs, from 0 hr to 24 hrs, from 0 hr to 10 hrs, from 0 hr to 5 hrs, or from 0 hr to 2 hrs. The culture medium may include any suitable factors to promote the growth and expansion of HSPCs, as described above.

In some cases, the in vitro mixed population includes a population of HSCs having at least one corrected SCD-associated SNP that remains corrected for a period of time after contacting the in vitro mixed population with the genome editing composition. The period of time may be for at least one month following said contacting, for at least 6 months following said contacting, for at least 1 year following said contacting, or for at least 2 years following said contacting. The at least one corrected SCD-associated SNP may remain permanently corrected after said contacting. In some cases, 2% to 20% of HSCs in the in vitro mixed population comprise at least one corrected SCD-associated SNP that remains corrected for a period of time; for example, 2% to 25% of HSCs, 2% to 30% of HSCs, 2% to 35% of HSCs, 2% to 40% of HSCs, 2% to 45% of HSCs, 2% to 50%, or 50% or more of HSCs in the in vitro population comprise at least one corrected SCD-associated SNP that remains corrected for a period of time after said administering.

Aspects of the methods further include cryopreserving the in vitro mixed population after the contacting with the genome editing composition has occurred, e.g., after genome editing has occurred, after correction of the SCD-associated SNP has occurred, etc. In some cases, the in vitro mixed population may be cryopreserved from 0 hr to 30 hr after the contacting has occurred; for example, the in vitro mixed population may be cryopreserved from 0 hr to 24 hr, from 0 hr to 12 hr, or from 0 hr to 6 hr after the contacting has occurred. Any known method used to successfully cryopreserve. HSPCs may be applied. The in vitro mixed population may be preserved in any standard cryopreservation solution. Accordingly, using cryopreservation, the stem cells can be maintained such that once it is determined that a subject or individual is in need of stem cell transplantation, the stem cells can be thawed and transplanted back into the subject. The use of one or more. HSPC modulators, for example PGE2, during cryopreservation techniques may enhance the HSPC population.

In some cases, the cryopreserved cells are thawed just prior to administration to an individual in need thereof (e.g., an individual having SCD). For example, in some cases, the cryopreserved in vitro mixed population is thawed from 5 minutes to 4 hours (e.g., from 5 minutes to 10 minutes, from 10 minutes to 30 minutes, from 30 minutes to 60 minutes, from 1 hour to 2 hours, or from 2 hours to 4 hours) prior to administration to an individual in need thereof (e.g., all individual having SCD).

### CRISPR Enzymes

A CRISPR enzyme suitable for inclusion in the methods of the present disclosure includes an RNA-guided endonuclease, also referred to herein as a “genome-editing nuclease.” The CRISPR enzyme may be a Class 2 CRISPR effector protein, also referred to herein as a class 2 CRISPR/Cas effector polypeptide.

Examples of RNA-guided endonucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some cases, a suitable RNA-guided endonuclease is a class 2 CRISPR/Cas endonuclease. In some cases, a suitable RNA-guided endonuclease is a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a suitable RNA-guided endonuclease is a class 2 type VI CRISPR/Cas endonuclease (e.g., a C2c2 protein; also referred to as a “Cas13a” protein). Also suitable for use is a CasX protein. Also suitable for use is a CasY protein.

In some cases, the genome-editing endonuclease is a CasX or a CasY polypeptide. CasX and CasY polypeptides are described in Burstein et al. (2017) Nature 542:237.

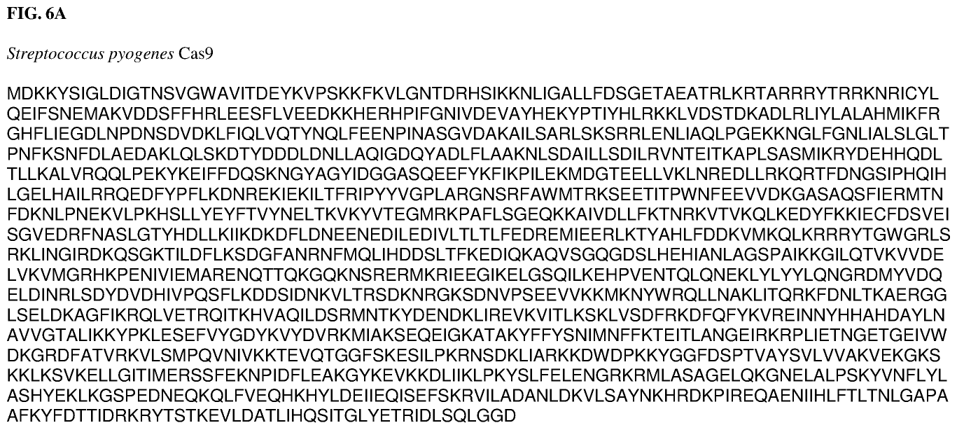

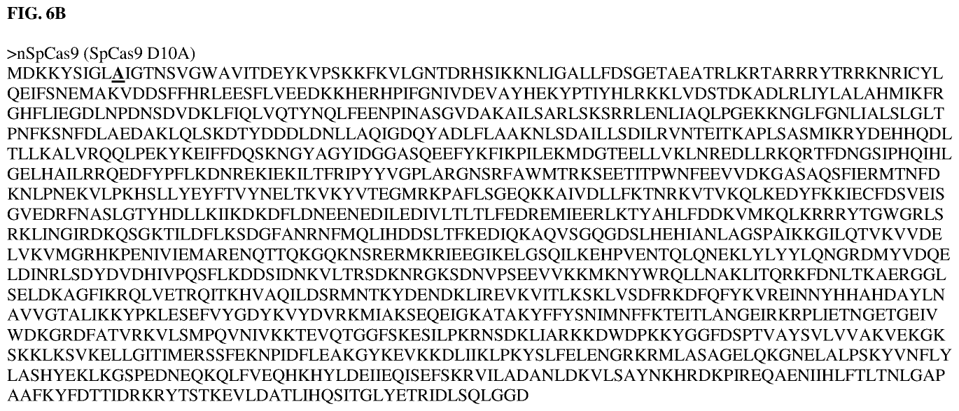

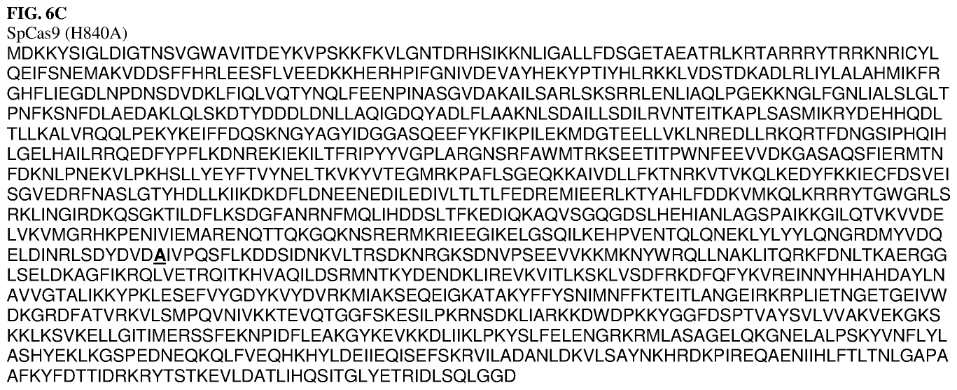

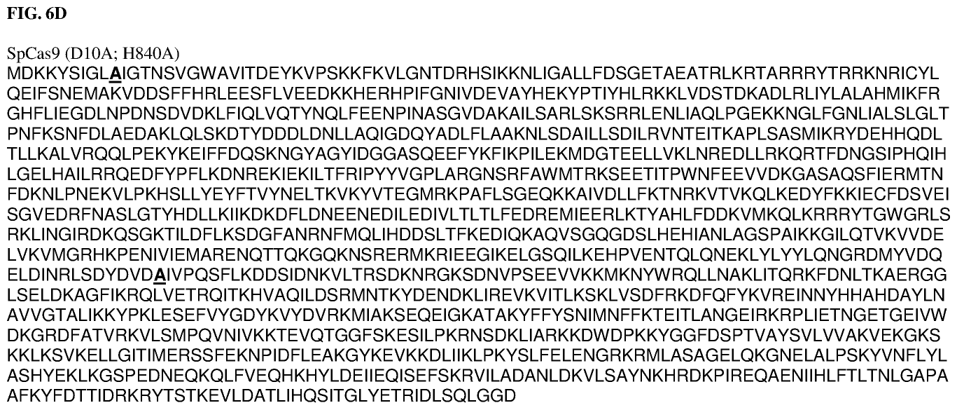

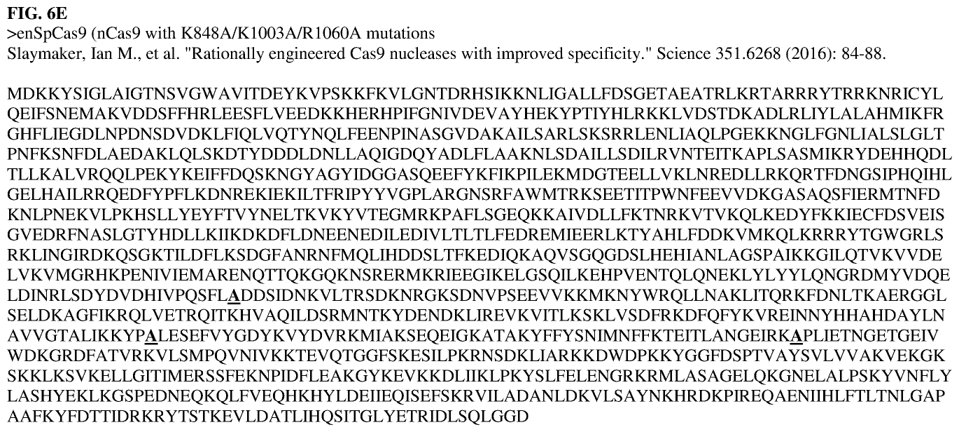

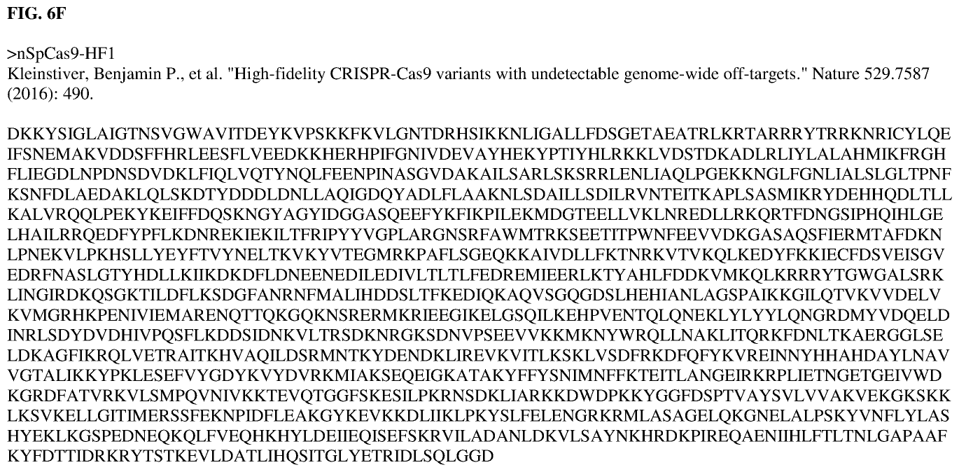

In some cases, the genome-editing endonuclease is a Type II CRISPR/Cas endonuclease. In some cases, the genome-editing endonuclease is a Cas9 polypeptide, also referred to herein as a “Cas9 enzyme.” The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 6A. In some cases, a Cas9 polypeptide comprises the amino acid sequence depicted in one of FIG. 6A-6F.

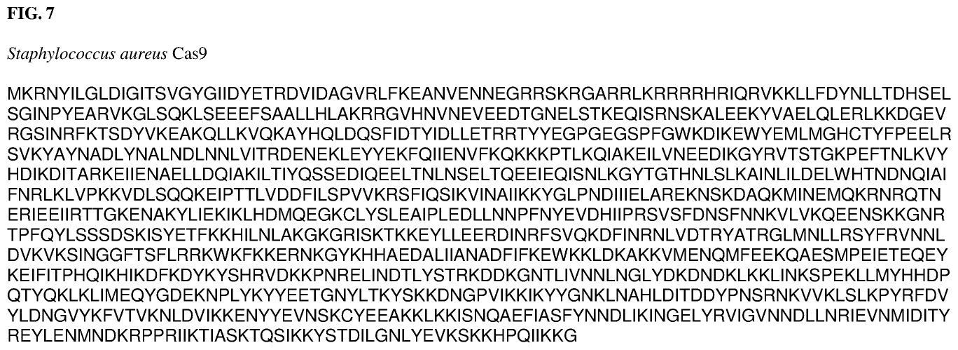

In some cases, the Cas9 polypeptide used in a composition or method of the present disclosure is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 7.

In some cases, the Cas9 polypeptide used in a composition or method of the present disclosure is a Campylobacter jejuni Cas9 (CjCas9) polypeptide. CjCas9 recognizes the 5′-NNNVRYM-3′ as the protospacer-adjacent motif (PAM). One example of an amino acid sequence of CjCas9 is set forth in SEQ ID NO:50. In some cases, a Cas9 polypeptide suitable for use in a composition or method of the present disclosure comprises an amino acid sequence having at least 50%, at least 60%, at least 70%, at least 80%, at least 90%, at least 95%, at least 98%, at least 99%, or more than 99%, amino acid sequence identity to a CjCas9 amino acid sequence (e.g., the CjCas9 amino acid sequence set forth in SEQ ID NO:50).

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 6A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 6A, where amino acids N497, R661, Q695, and Q926 are substituted, e.g., with alanine.

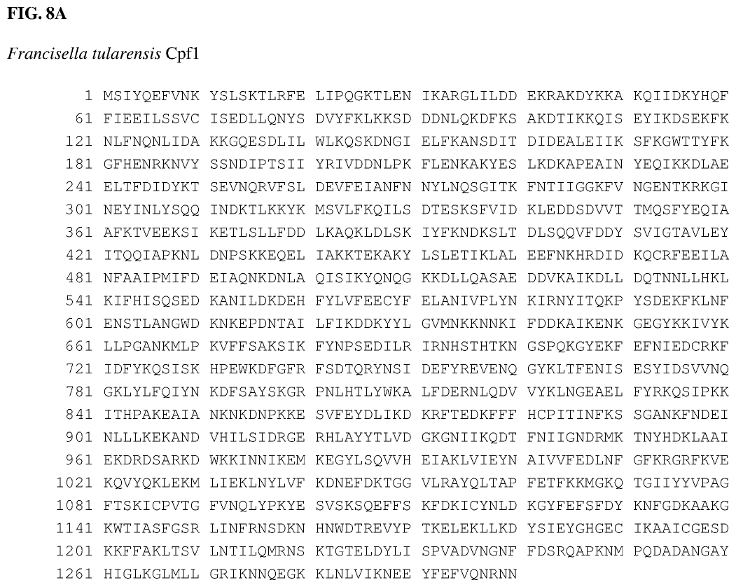

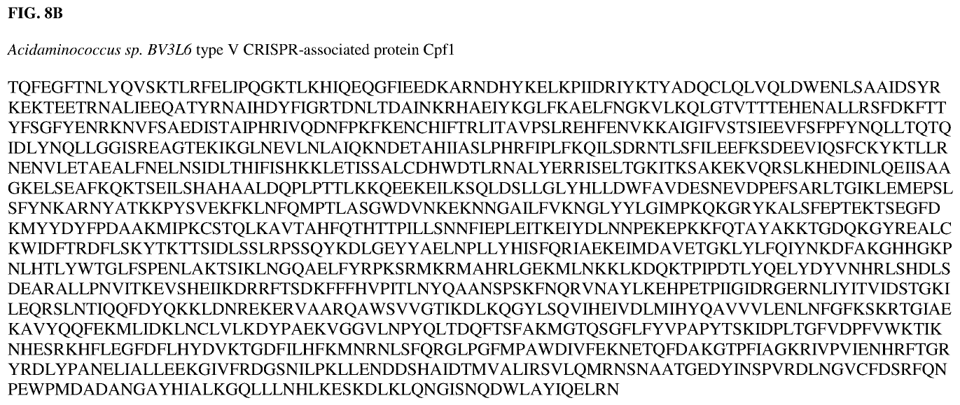

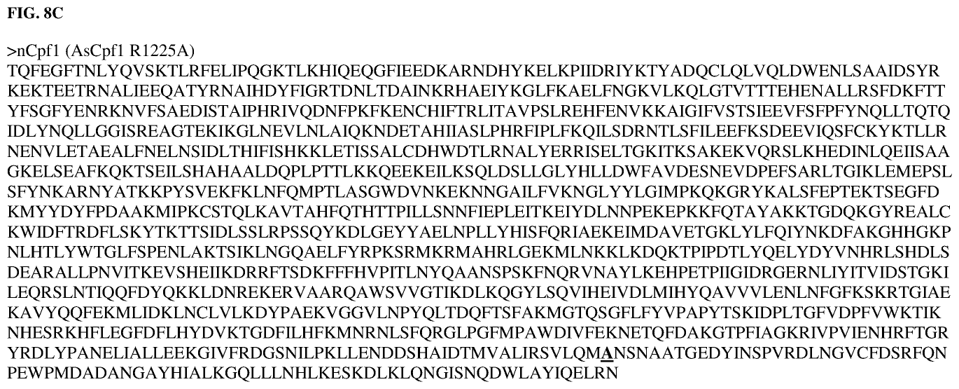

In some cases, the genome-editing endonuclease is a type V CRISPR/Cas endonuclease. In some cases a type V CRISPR/Cas endonuclease is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 8A, FIG. 8B, or FIG. 8C.

In some cases, a suitable Cas9 polypeptide exhibits altered PAM specificity. See, e.g., Kleinstiver et al. (2015) Nature 523:481.

RNA-Guided Endonucleases

An RNA-guided endonuclease is also referred to herein as a “genome editing nuclease.” Examples of suitable genome editing nucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some cases, a genome targeting composition includes a class 2 CRISPR/Cas endonuclease. In some cases, a genome targeting composition includes a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a genome targeting composition includes a class 2 type VI CRISPR/Cas endonuclease (e.g., a C2c2 protein; also referred to as a “Cas13a” protein). Also suitable for use is a CasX protein. Also suitable for use is a CasY protein.

In some cases, a genome editing nuclease is a fusion protein that is fused to a heterologous polypeptide (also referred to as a “fusion partner”). In some cases, a genome editing nuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.).

In some cases, the genome-editing endonuclease is a Type II CRISPR/Case endonuclease. In some cases, the genome-editing endonuclease is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 6A. In some cases, the Cas9 polypeptide used in a composition or method of the present disclosure is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 7.

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 6A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 6A, where amino acids N497, R661, Q695, and Q926 are substituted, e.g., with alanine.

In some cases, a suitable Cas9 polypeptide exhibits altered PAM specificity. See, e.g., Kleinstiver et al. (2015) Nature 523:481.

In some cases, the genome-editing endonuclease is a type V CRISPR/Cas endonuclease. In some cases a type V CRISPR/Cas endonuclease is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 8A. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 8B. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 8C.

A nucleic acid that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, a composition of the present disclosure comprises an RNA-guided endonuclease, or both an RNA-guided endonuclease and a guide RNA. In some cases, e.g., where a target nucleic acid comprises a deleterious mutation in a defective allele (e.g., a deleterious mutation in a retinal cell target nucleic acid), the RNA-guided endonuclease/guide RNA complex, together with a donor nucleic acid comprising a nucleotide sequence that corrects the deleterious mutation (e.g., a donor nucleic acid comprising a nucleotide sequence that encodes a functional copy of the protein encoded by the defective allele), can be used to correct the deleterious mutation, e.g., via homology-directed repair (HDR).

In some cases, a composition of the present disclosure comprises: i) an RNA-guided endonuclease; and ii) one guide RNA. In some cases, the guide RNA is a single-molecule (or “single guide”) guide RNA (a “sgRNA”). In some cases, the guide RNA is a dual-molecule (or “dual-guide”) guide RNA (“dgRNA”).

In some cases, a composition of the present disclosure comprises: i) an RNA-guided endonuclease; and ii) 2 separate sgRNAs, where the 2 separate sgRNAs provide for deletion of a target nucleic acid via non-homologous end joining (NHEJ). In some cases, the guide RNAs are sgRNAs. In some cases, the guide RNAs are dgRNAs.

In some cases, a composition of the present disclosure comprises: i) a Cpf1 polypeptide; and ii) a guide RNA precursor; in these cases, the precursor can be cleaved by the Cpf1 polypeptide to generate 2 or more guide RNAs.

Class 2 CRISPR/Cas Endonucleases

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In class 2 CRISPR systems, the functions of the effector complex (e.g., the cleavage of target DNA) are carried out by a single endonuclease (e.g., see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97); and Shmakov et al. (2017) Nature Reviews Microbiology 15:169. As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9); type V-A CRISPR/Cas proteins (e.g., Cpf1 (also referred to a “Cas12a”)); type V-B CRISPR/Cas proteins (e.g., C2c1 (also referred to as “Cas12b”)); type V-C CRISPR/Cas proteins (e.g., C2c3 (also referred to as “Cas12c”)); type V-U1 CRISPR/Cas proteins (e.g., C2c4); type V-U2 CRISPR/Cas proteins (e.g., C2c8); type V-U5 CRISPR/Cas proteins (e.g., C2c5); type V-U4 CRISPR/Cas proteins (e.g., C2c9); type V-U3 CRISPR/Cas proteins (e.g., C2c10); type VI-A CRISPR/Cas proteins (e.g., C2c2 (also known as “Cas13a”)); type VI-B CRISPR/Cas proteins (e.g., Cas13b (also known as C2c4)); and type VI-C CRISPR/Cas proteins (e.g., Cas13c (also known as C2c7)). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex.

Type II CRISPR/Cas Endonucleases (e.g., Cas 9)

In natural Type II CRISPR/Cas systems, Cas9 functions as an RNA-guided endonuclease that uses a dual-guide RNA having a crRNA and trans-activating crRNA (tracrRNA) for target recognition and cleavage by a mechanism involving two nuclease active sites in Cas9 that together generate double-stranded DNA breaks (DSBs), or can individually generate single-stranded DNA breaks (SSBs). The Type II CRISPR endonuclease Cas9 and engineered dual-(dgRNA) or single guide RNA (sgRNA) form a ribonucleoprotein (RNP) complex that can be targeted to a desired DNA sequence.

A type II CRISPR/Cas endonuclease is a type of class 2 CRISPR/Cas endonuclease. In some cases, the type II CRISPR/Cas endonuclease is a Cas9 protein. A Cas9 protein forms a complex with a Cas9 guide RNA. The guide RNA provides target specificity to a Cas9-guide RNA complex by having a nucleotide sequence (a guide sequence) that is complementary to a sequence (the target site) of a target nucleic acid (as described elsewhere herein). The Cas9 protein of the complex provides the site-specific activity. In other words, the Cas9 protein is guided to a target site (e.g., stabilized at a target site) within a target nucleic acid sequence (e.g. a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA.

A Cas9 protein can bind and/or modify (e.g., cleave, nick, methylate, demethylate, etc.) a target nucleic acid and/or a polypeptide associated with target nucleic acid (e.g., methylation or acetylation of a histone tail)(e.g., when the Cas9 protein includes a fusion partner with an activity). In some cases, the Cas9 protein is a naturally-occurring protein (e.g., naturally occurs in bacterial and/or archaeal cells). In other cases, the Cas9 protein is not a naturally-occurring polypeptide (e.g., the Cas9 protein is a variant Cas9 protein).

Examples of suitable Cas9 proteins include, but are not limited to, those set forth in SEQ ID NOs: 5-816. Naturally occurring Cas9 proteins bind a Cas9 guide RNA, are thereby directed to a specific sequence within a target nucleic acid (a target site), and cleave the target nucleic acid (e.g., cleave dsDNA to generate a double strand break, cleave ssDNA, cleave ssRNA, etc.).

Assays to determine whether given protein interacts with a Cas9 guide RNA can be any convenient binding assay that tests for binding between a protein and a nucleic acid. Suitable binding assays (e.g., gel shift assays) will be known to one of ordinary skill in the art (e.g., assays that include adding a Cas9 guide RNA and a protein to a target nucleic acid).

Assays to determine whether a protein has an activity (e.g., to determine if the protein has nuclease activity that cleaves a target nucleic acid and/or some heterologous activity) can be any convenient assay (e.g., any convenient nucleic acid cleavage assay that tests for nucleic acid cleavage). Suitable assays (e.g., cleavage assays) will be known to one of ordinary skill in the art and can include adding a Cas9 guide RNA and a protein to a target nucleic acid.

Many Cas9 orthologs from a wide variety of species have been identified and in some cases the proteins share only a few identical amino acids. Identified Cas9 orthologs have similar domain architecture with a central HNH endonuclease domain and a split RuvC/RNaseH domain (e.g., RuvCI, RuvCII, and RuvCIII) (e.g., see Table 1). For example, a Cas9 protein can have 3 different regions (sometimes referred to as RuvC-I, RuvC-II, and RucC-III), that are not contiguous with respect to the primary amino acid sequence of the Cas9 protein, but fold together to form a RuvC domain once the protein is produced and folds. Thus, Cas9 proteins can be said to share at least 4 key motifs with a conserved architecture. Motifs 1, 2, and 4 are RuvC like motifs while motif 3 is an HNH-motif. The motifs set forth in Table 1 may not represent the entire RuvC-like and/or HNH domains as accepted in the art, but Table 1 does present motifs that can be used to help determine whether a given protein is a Cas9 protein.

TABLE 1
Table 1 lists 4 motifs that are present in Cas9 sequences
from various species. The amino acids listed in Table 1
are from the Cas9 from S. pyogenes (SEQ ID NO: 5).

| Motif # | Motif | Amino acids (residue #s) | Highly conserved |
|---|---|---|---|
| 1 | RuvC-like I | IGLDIGTNSVGWAVI (7-21) | D10, G12, G17 |
| | | (SEQ ID NO: 1) | |
| 2 | RuvC-like II | IVIEMARE (759-766) | E762 |
| | | (SEQ ID NO: 2) | |
| 3 | HNH-motif | DVDHIVPQSFLKDDSIDNKVLTRSDK | H840, N854, N863 |
| | | N (837-863) (SEQ ID NO: 3) | |
| 4 | RuvC-like | HHAHDAYL (982-989) | H982, H983, A984, |
| | III | (SEQ ID NO: 4) | D986, A987 |

In some cases, a suitable Cas9 protein comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 as set forth in SEQ ID NOs: 1-4, respectively (e.g., see Table 1), or to the corresponding portions in any of the amino acid sequences set forth in SEQ ID NOs: 5-816.

In other words, in some cases, a suitable Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 of the Cas9 amino acid sequence set forth in SEQ ID NO: 5 (e.g., the sequences set forth in SEQ ID NOs: 1-4, e.g., see Table 1), or to the corresponding portions in any of the amino acid sequences set forth in SEQ ID NOs: 6-816.

In some cases, a suitable Cas9 protein comprises an amino acid sequence having 4 motifs, each of motifs 1-4 having 60% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 70% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 75% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 80% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 85% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 90% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 95% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 99% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 100% amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816.

In some cases, a suitable Cas9 protein comprises an amino acid sequence having 60% or more, 70% or more, 75% or more, 80% or more, 85% or more, 90% or more, 95% or more, 99% or more or 100% amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816.

Examples of various Cas9 proteins (and Cas9 domain structure) and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; Shmakov et al., Nat Rev Microbiol. 2017 March; 15(3):169-182; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

Type V and Type VI CRISPR/Cas Endonucleases

In some cases, a genome targeting composition of the present disclosure includes a type V or type VI CRISPR/Cas endonuclease (i.e., the genome editing endonuclease is a type V or type VI CRISPR/Cas endonuclease) (e.g., Cpf1, C2c1, C2c2, C2c3). Type V and type VI CRISPR/Cas endonucleases are a type of class 2 CRISPR/Cas endonuclease. Examples of type V CRISPR/Cas endonucleases include but are not limited to: Cpf1, C2c1, and C2c3. An example of a type VI CRISPR/Cas endonuclease is C2c2. In some cases, a subject genome targeting composition includes a type V CRISPR/Cas endonuclease (e.g., Cpf1, C2c1, C2c3). In some cases, a Type V CRISPR/Cas endonuclease is a Cpf1 protein. In some cases, a subject genome targeting composition includes a type VI CRISPR/Cas endonuclease (e.g., Cas13a).

Like type II CRISPR/Cas endonucleases, type V and VI CRISPR/Cas endonucleases form a complex with a corresponding guide RNA. The guide RNA provides target specificity to an endonuclease-guide RNA RNP complex by having a nucleotide sequence (a guide sequence) that is complementary to a sequence (the target site) of a target nucleic acid (as described elsewhere herein). The endonuclease of the complex provides the site-specific activity. In other words, the endonuclease is guided to a target site (e.g., stabilized at a target site) within a target nucleic acid sequence (e.g. a chromosomal sequence or an extrachromosomal sequence, e.g., an episomal sequence, a minicircle sequence, a mitochondrial sequence, a chloroplast sequence, etc.) by virtue of its association with the protein-binding segment of the guide RNA.

Examples and guidance related to type V and type VI CRISPR/Cas proteins (e.g., Cpf1, C2c1, C2c2, and C2c3 guide RNAs) can be found in the art, for example, see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97; and Shmakov et al. (2017) Nature Reviews Microbiology 15:169.

In some cases, the Type V or type VI CRISPR/Cas endonuclease (e.g., Cpf1, C2c1, C2c2, C2c3) is enzymatically active, e.g., the Type V or type VI CRISPR/Cas polypeptide, when bound to a guide RNA, cleaves a target nucleic acid.

In some cases a type V CRISPR/Cas endonuclease is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs: 818-822. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the Cpf1 amino acid sequence set forth in any of SEQ ID NOs:818-822.

In some cases a type V CRISPR/Cas endonuclease is a C2c1 protein (examples include those set forth as SEQ ID NOs: 823-830). In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c1 amino acid sequence set forth in any of SEQ ID NOs: 823-830. In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c1 amino acid sequence set forth in any of SEQ ID NOs: 823-830.

In some cases a type V CRISPR/Cas endonuclease is a C2c3 protein (examples include those set forth as SEQ ID NOs: 831-834). In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c3 amino acid sequence set forth in any of SEQ ID NOs: 831-834. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c3 amino acid sequence set forth in any of SEQ ID NOs: 831-834.

In some cases a type VI CRISPR/Cas endonuclease is a C2c2 protein (examples include those set forth as SEQ ID NOs: 835-846). In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c2 amino acid sequence set forth in any of SEQ ID NOs: 835-846. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c2 amino acid sequence set forth in any of SEQ ID NOs: 835-846.

Examples and guidance related to type V or type VI CRISPR/Cas endonucleases (including domain structure) and guide RNAs (as well as information regarding requirements related to protospacer adjacent motif (PAM) sequences present in targeted nucleic acids) can be found in the art, for example, see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97; and Shmakov et al., Nat Rev Microbiol. 2017 March; 15(3):169-182; and U.S. patents and patent applications: U.S. Pat. No. 9,580,701; 20170073695, 20170058272, 20160362668, 20160362667, 20160298078, 20160289637, 20160215300, 20160208243, and 20160208241, each of which is hereby incorporated by reference in its entirety.

CasX and CasY Proteins

Suitable RNA-guided endonucleases include CasX and CasY proteins. See, e.g., Burstein et al. (2017) Nature 542:237.

Nucleic Acid Modifications

In some embodiments, a subject nucleic acid (e.g., a guide RNA) has one or more modifications, e.g., a base modification, a backbone modification, a sugar modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

The guide RNA of the subject methods may include one or more modifications at or near the 5′ end. In some cases, the first three nucleotides at the 5′ end and/or the 3′ end of the guide RNA include nucleic acid modifications. In some instances, nucleic acid modifications at the 5′ end and/or the 3′ end of the guide RNA include three 2′-OMe 3′-phosphorothioates (3×MS).

Suitable nucleic acid modifications include, but are not limited to: 2′Omethyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

In some cases, 2% or more of the nucleotides of a nucleic acid (e.g., a guide RNA, etc.) are modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are modified). In some cases, 2% or more of the nucleotides of a subject nucleic acid are modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are modified). In some cases, 2% or more of the nucleotides of a nucleic acid are modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are modified).

In some cases, the number of nucleotides of a subject nucleic acid nucleic acid (e.g., a guide RNA etc.) that are modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject that are modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that are modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%).

In some cases, one or more of the nucleotides of a nucleic acid (e.g., a guide RNA, etc.) are modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are modified). In some cases, one or more of the nucleotides of a subject nucleic acid are modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are modified). In some cases, one or more of the nucleotides of a subject nucleic acid are modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are modified).

In some cases, 99% or less of the nucleotides of a nucleic acid (e.g., a guide RNA, etc.) are modified (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are modified). In some cases, 99% or less of the nucleotides of a subject nucleic acid are modified (e.g., e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are modified). In some cases, 99% or less of the nucleotides of a subject nucleic acid are modified (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are modified).

In some cases, the number of nucleotides of a nucleic acid (e.g., a guide RNA, etc.) that are modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that are modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that are modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10).

In some cases, 20 or fewer of the nucleotides of a nucleic acid (e.g., a guide RNA, etc.) are modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are modified). In some cases, 20 or fewer of the nucleotides of a subject nucleic acid are modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are modified). In some cases, 20 or fewer of the nucleotides of a subject nucleic acid are modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are modified).

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stable with respect to attack by single-stranded ribonucleases and is typically 5 to 10-fold less susceptible to DNases than DNA. It is commonly used in antisense oligos as a means to increase stability and binding affinity to the target message.

In some cases, 2% or more of the nucleotides of a nucleic acid (e.g., a guide RNA, etc.) are 2′-O-Methyl modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are 2′-O-Methyl modified). In some cases, 2% or more of the nucleotides of a subject nucleic acid are 2′-O-Methyl modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are 2′-O-Methyl modified). In some cases, 2% or more of the nucleotides of a subject nucleic acid are 2′-O-Methyl modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are 2′-O-Methyl modified).

In some cases, the number of nucleotides of a nucleic acid nucleic acid (e.g., a guide RNA, etc.) that are 2′-O-Methyl modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that are 2′-O-Methyl modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that are 2′-O-Methyl modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%).

In some cases, one or more of the nucleotides of a nucleic acid (e.g., a guide RNA, etc.) are 2′-O-Methyl modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are 2′-O-Methyl modified). In some cases, one or more of the nucleotides of a subject nucleic acid are 2′-O-Methyl modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are 2′-O-Methyl modified). In some cases, one or more of the nucleotides of a subject nucleic acid are 2′-O-Methyl modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are 2′-O-Methyl modified).

In some cases, 99% or less of the nucleotides of a nucleic acid (e.g., a guide RNA, etc.) are 2′-O-Methyl modified (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are 2′-O-Methyl modified). In some cases, 99% or less of the nucleotides of a subject nucleic acid are 2′-O-Methyl modified (e.g., e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are 2′-O-Methyl modified). In some cases, 99% or less of the nucleotides of a subject nucleic acid are 2′-O-Methyl modified (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are 2′-O-Methyl modified).

In some cases, the number of nucleotides of a nucleic acid nucleic acid (e.g., a guide RNA, etc.) that are 2′-O-Methyl modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that are 2′-O-Methyl modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that are 2′-O-Methyl modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10).

In some cases, 20 or fewer of the nucleotides of a nucleic acid (e.g., a guide RNA, etc.) are 2′-O-Methyl modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are 2′-O-Methyl modified). In some cases, 20 or fewer of the nucleotides of a subject nucleic acid are 2′-O-Methyl modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are 2′-O-Methyl modified). In some cases, 20 or fewer of the nucleotides of a subject nucleic acid are 2′-O-Methyl modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are 2′-O-Methyl modified).

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

In some cases, 2% or more of the nucleotides of a nucleic acid (e.g., a guide RNA, etc.) are 2′ Fluoro modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are 2′ Fluoro modified). In some cases, 2% or more of the nucleotides of a subject nucleic acid are 2′ Fluoro modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are 2′ Fluoro modified). In some cases, 2% or more of the nucleotides of a subject nucleic acid are 2′ Fluoro modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are 2′ Fluoro modified).

In some cases, the number of nucleotides of a nucleic acid nucleic acid (e.g., a guide RNA, etc.) that are 2′ Fluoro modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that are 2′ Fluoro modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that are 2′ Fluoro modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%).

In some cases, one or more of the nucleotides of a nucleic acid (e.g., a guide RNA, etc.) are 2′ Fluoro modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are 2′ Fluoro modified). In some cases, one or more of the nucleotides of a subject nucleic acid are 2′ Fluoro modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are 2′ Fluoro modified). In some cases, one or more of the nucleotides of a subject nucleic acid are 2′ Fluoro modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid are 2′ Fluoro modified).

In some cases, 99% or less of the nucleotides of a nucleic acid (e.g., a guide RNA, etc.) are 2′ Fluoro modified (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are 2′ Fluoro modified). In some cases, 99% or less of the nucleotides of a subject nucleic acid are 2′ Fluoro modified (e.g., e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are 2′ Fluoro modified). In some cases, 99% or less of the nucleotides of a subject nucleic acid are 2′ Fluoro modified (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid are 2′ Fluoro modified).

In some cases, the number of nucleotides of a nucleic acid nucleic acid (e.g., a guide RNA, etc.) that are 2′ Fluoro modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that are 2′ Fluoro modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that are 2′ Fluoro modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10).

In some cases, 20 or fewer of the nucleotides of a nucleic acid (e.g., a guide RNA, etc.) are 2′ Fluoro modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are 2′ Fluoro modified). In some cases, 20 or fewer of the nucleotides of a subject nucleic acid are 2′ Fluoro modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are 2′ Fluoro modified). In some cases, 20 or fewer of the nucleotides of a subject nucleic acid are 2′ Fluoro modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid are 2′ Fluoro modified).

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In some cases, the number of LNAs incorporated into a single oligo is 10 bases or less.

In some cases, the number of nucleotides of a nucleic acid nucleic acid (e.g., a guide RNA, etc.) that have an LNA base is in a range of from 3% to 99% (e.g., 3% to 99%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 99%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that have an LNA base is in a range of from 3% to 99% (e.g., 3% to 99%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 99%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that have an LNA base is in a range of from 3% to 99% (e.g., 3% to 99%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 99%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%).

In some cases, one or more of the nucleotides of a nucleic acid (e.g., a guide RNA, etc.) have an LNA base (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid have an LNA base). In some cases, one or more of the nucleotides of a subject nucleic acid have an LNA base (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid have an LNA base). In some cases, one or more of the nucleotides of a subject nucleic acid have an LNA base (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid have an LNA base).

In some cases, 99% or less of the nucleotides of a nucleic acid (e.g., a guide RNA, etc.) have an LNA base (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid have an LNA base). In some cases, 99% or less of the nucleotides of a subject nucleic acid have an LNA base (e.g., e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid have an LNA base). In some cases, 99% or less of the nucleotides of a subject nucleic acid have an LNA base (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid have an LNA base).

In some cases, the number of nucleotides of a nucleic acid nucleic acid (e.g., a guide RNA, etc.) that have an LNA base is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that have an LNA base is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a subject nucleic acid that have an LNA base is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10).

In some cases, 20 or fewer of the nucleotides of a nucleic acid (e.g., a guide RNA, etc.) have an LNA base (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid have an LNA base). In some cases, 20 or fewer of the nucleotides of a subject nucleic acid have an LNA base (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid have an LNA base). In some cases, 20 or fewer of the nucleotides of a subject nucleic acid have an LNA base (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid have an LNA base).

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some cases, the number of nucleotides of a nucleic acid nucleic acid (e.g., a guide RNA, etc.) that have a phosphorothioate linkage is in a range of from 3% to 99% (e.g., 3% to 99%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 99%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that have a phosphorothioate linkage is in a range of from 3% to 99% (e.g., 3% to 99%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 99%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a subject nucleic acid that have a phosphorothioate linkage is in a range of from 3% to 99% (e.g., 3% to 99%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 99%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%).

In some cases, one or more of the nucleotides of a nucleic acid (e.g., a guide RNA, etc.) have a phosphorothioate linkage (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid have a phosphorothioate linkage). In some cases, one or more of the nucleotides of a subject nucleic acid have a phosphorothioate linkage (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a subject nucleic acid have a phosphorothioate linkage). In some cases, one or more of the nucleotides of a nucleic acid have a phosphorothioate linkage (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a nucleic acid have a phosphorothioate linkage).

In some cases, 99% or less of the nucleotides of a nucleic acid (e.g., a guide RNA, etc.) have a phosphorothioate linkage (e.g., 99% or less, 95% or less, 90% or less, 85% or less, 80% or less, 75% or less, 70% or less, 65% or less, 60% or less, 55% or less, 50% or less, or 45% or less of the nucleotides of a subject nucleic acid have a phosphorothioate linkage). In some cases, 99% or less of the nucleotides of a subject nucleic acid have a phosphorothioate linkage (e.g., e.g., 99% or less, 95% or less, 90% or less, 85% or less, 80% or less, 75% or less, 70% or less, 65% or less, 60% or less, 55% or less, 50% or less, or 45% or less of the nucleotides of a nucleic acid have a phosphorothioate linkage). In some cases, 99% or less of the nucleotides of a nucleic acid have a phosphorothioate linkage (e.g., 99% or less, 95% or less, 90% or less, 85% or less, 80% or less, 75% or less, 70% or less, 65% or less, 60% or less, 55% or less, 50% or less, or 45% or less of the nucleotides of a nucleic acid have a phosphorothioate linkage).

In some cases, the number of nucleotides of a nucleic acid nucleic acid (e.g., a guide RNA, etc.) that have a phosphorothioate linkage is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a nucleic acid that have a phosphorothioate linkage is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a nucleic acid that have a phosphorothioate linkage is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10).

In some cases, 20 or fewer of the nucleotides of a nucleic acid (e.g., a guide RNA, etc.) have a phosphorothioate linkage (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid have a phosphorothioate linkage). In some cases, 20 or fewer of the nucleotides of a nucleic acid have a phosphorothioate linkage (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a subject nucleic acid have a phosphorothioate linkage). In some cases, 20 or fewer of the nucleotides of a nucleic acid have a phosphorothioate linkage (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a nucleic acid have a phosphorothioate linkage).

In some cases, a nucleic acid (e.g., a guide RNA, etc.) has one or more nucleotides that are 2′-O-Methyl modified nucleotides. In some embodiments, a subject nucleic acid (e.g., a guide RNA, etc.) has one or more 2′ Fluoro modified nucleotides. In some cases, a subject nucleic acid (e.g., a guide RNA, etc.) has one or more LNA bases. In some cases, a subject nucleic acid (e.g., a guide RNA, etc.) has one or more nucleotides that are linked by a phosphorothioate bond (i.e., the subject nucleic acid has one or more phosphorothioate linkages). In some embodiments, a subject nucleic acid (e.g., a guide RNA, etc.) has a 5′ cap (e.g., a 7-methylguanylate cap (m7G)).

In some cases, a subject nucleic acid has a combination of modified nucleotides. For example, a nucleic acid can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage). A nucleic acid can have any combination of modifications. For example, a subject nucleic acid can have any combination of the above described modifications.

In some cases, a subject nucleic acid has one or more nucleotides that are 2′-O-Methyl modified nucleotides. In some embodiments, a subject nucleic acid has one or more 2′ Fluoro modified nucleotides. In some embodiments, a subject nucleic acid has one or more LNA bases. In some embodiments, a subject nucleic acid has one or more nucleotides that are linked by a phosphorothioate bond (i.e., the subject nucleic acid has one or more phosphorothioate linkages). In some embodiments, a subject nucleic acid has a 5′ cap (e.g., a 7-methylguanylate cap (m7G)).

In some cases, a subject nucleic acid has a combination of modified nucleotides. For example, a subject nucleic acid can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage). A subject nucleic acid can have any combination of modifications. For example, a subject nucleic acid can have any combination of the above described modifications.

Modified Backbones and Modified Internucleoside Linkages

Examples of suitable nucleic acids containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a nucleic acid comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Methods of Treating Sickle Cell Disease

The present disclosure provides a method of treating sickle cell disease (SCD) in an individual. The method may include a) modifying a globin gene in the genome of a hematopoietic stem/progenitor cell (HSPC) obtained from the individual according to any embodiment of the subject methods, thereby generating an in vitro mixed population, wherein at least 2%, e.g., at least 5%, at least 10%, at least 15%, at least 20%, at least 25%, at least 30%, at least 35%, at least 40%, at least 45%, at least 50%, or more than 50%, of the SCD-associated SNPs have been corrected in the in vitro mixed population; and b) administering the in vitro mixed population to the individual, thereby treating the SCD in the individual. The term “treated individual” as used herein may refer to an individual to whom an in vitro mixed population has been administered.

In some cases, the administering of the in vitro mixed population produces an engrafted population. The administering may include, e.g., infusing the in vitro mixed population into an individual, engrafting the in vitro mixed population into an individual, transplanting the in vitro mixed population into an individual, etc. The administering of the in vitro mixed population may occur after ablation of the bone marrow in an individual. By “engrafted population” is meant a population of transplanted cells such as a population of cells including, e.g., cells of the administered in vitro mixed population, cells derived from the administered in vitro mixed population, etc. The engrafted population may include population may include three populations of cells: 1) a population of cells that have two non-corrected β-globin alleles with SCD-associated SNPs; 2) a population of cells that have only one β-globin allele with an SCD-associated SNP that has been corrected; and 3) a population of cells that have two β-globin alleles with SCD-associated SNPs that have been corrected. In some cases, the population of cells having two non-corrected β-globin alleles includes cells where one or more β-globin alleles have been knocked out. The knockout of one or more β-globin alleles may be due to non-homologous end joining (NHEJ) where small insertions or deletions (indels) are inserted at the site of cleavage, where the indels cause functional disruption through introduction of non-specific mutations at the cleavage location. The engrafted population may include viable HSCs capable of long-term self-renewal. In some cases, the percentage of the β-globin alleles with SCD-associated SNPs that have been corrected in the engrafted population is at least 2%, e.g., at least 5%, at least 10%, at least 15%, at least 20%, at least 25%, at least 30%, at least 35%, at least 40%, at least 45%, at least 50%, or more than 50%. In some cases, at least 2% of the β-globin alleles with SCD-associated SNPs have been corrected; for example at least 2%, at least 5%, at least 10%, at least 15%, at least 20%, at least 25%, at least 30%, at least 35%, at least 40%, at least 45%, at least 50%, or more than 50%, of the β-globin alleles in the engrafted population have a corrected SCD-associated SNP. A β-globin allele with a “corrected SCD-associated SNP” encodes a polypeptide subunit for forming HbA (and not HbS).

The corrected SCD alleles in the in vitro mixed population may be maintained in the engrafted population after administering the in vitro mixed population to an individual. The administering may include infusing any suitable dose or effective amount of the in vitro mixed population, e.g., a dose suitable to produce an engrafted population into an individual. In certain embodiments, the administering may include a dose of any suitable amount of an in vitro mixed population, e.g., a thawed in vitro mixed population previously cryopreserved, as described above. In some cases, a single dose of the in vitro mixed population is administered. In some cases, the method includes administering an effective amount of at least 10⁶ to 10⁹ cells from the in vitro mixed population, e.g., CD34⁺ HSPCs, per kilogram of body weight of the individual, such as, e.g., at least 10⁶ to 10⁷ cells from the in vitro mixed population per kilogram of body weight of the individual. In some cases, the method includes administering an effective amount of cells/kg ranging from 1.5×10⁶ to 1×10⁷ cells from the in vitro mixed population/kg of body weight, 2×10⁶ cells from the in vitro mixed population/kg of body weight to 3×10⁶ cells from the in vitro mixed population/kg of body weight, or 5×10⁶ cells from the in vitro mixed population/kg to 1×10⁷ cells from the in vitro mixed population/kg of body weight. In some cases, from about 0.5×10⁶ cells/kg to about 20×10⁶ cells/kg are harvested from a patient; these harvested cells are used to generate an in vitro mixed population, suitable for re-introduction into the patient, of about 3×10⁶ cells/kg.

Any suitable percentage of cells, e.g., bone marrow cells, in the engrafted population may have zero, one, or two corrected SCD-associated SNPs after a period of time, e.g., after the administering of the in vitro mixed population to an individual, and/or any suitable percentage of the total SCD-associated SNPs may be corrected after a period of time. The period of time may range from 1 day to 6 months after administration, from 6 months to 12 months after administration, from 1 year to 2 years after administration, or for a period of time after administration that lasts up to the years in the individual's lifespan. In some cases, the period of time is at least one month following said administering, at least 6 months following said administering, at least 1 year following said administering, or at least 2 years following said administering. In certain embodiments, 2% to 95% of cells of the engrafted population comprise two non-corrected SCD-associated SNPs after a period of time after said administering such as, e.g., 2% to 80% of cells, 2% to 70% of cells, 2% to 60% of cells, 2% to 50% of cells, 2% to 40% of cells, 2% to 30%, or 2% to 20% of cells. In certain embodiments, 2% to 95% of cells of the engrafted population comprise only one corrected SCD-associated SNP after a period of time after said administering such as, e.g., 2% to 80% of cells, 2% to 70% of cells, 2% to 60% of cells, 2% to 50% of cells, 2% to 40% of cells, 2% to 30%, or 2% to 20% of cells. The one corrected SCD-associated SNP may remain corrected for a period of time after engraftment up to the lifespan of the individual. In certain embodiments, 2% to 95% of cells of the engrafted population comprise two corrected SCD-associated SNPs after a period of time after said administering such as, e.g., 2% to 80% of cells, 2% to 70% of cells, 2% to 60% of cells, 2% to 50% of cells, 2% to 40% of cells, 2% to 30%, or 2% to 20% of cells. The two corrected SCD-associated SNPs may remain corrected for a period of time after engraftment up to the lifespan of the individual. In certain embodiments, 2% to 95% of cells from the engrafted population comprise at least one corrected SCD-associated SNP after a period of time after said administering such as, e.g., 2% to 80% of cells, 2% to 70% of cells, 2% to 60% of cells, 2% to 50% of cells, 2% to 40% of cells, 2% to 30%, or 2% to 20% of cells. In some cases, 20% of the engrafted population comprises at least one corrected SCD-associated SNP after a period of time ranging from 1 day to 6 months after administration, from 6 months to 12 months after administration, from 1 year to 2 years after administration, up to the years in the individual's lifespan. In some cases, the period of time is at least one month following said administering, at least 6 months following said administering, at least 1 year following said administering, or at least 2 years following said administering. In some cases, 0% to 95% of the SCD-associated SNPs in the engrafted population are corrected after a period of time after said administering such as, e.g., 0% to 80% of the SCD-associated SNPs, 0% to 70% of the SCD-associated SNPs, 0% to 60% of the SCD-associated SNPs, 0% to 50% of the SCD-associated SNPs, 0% to 40% of the SCD-associated SNPs, 0% to 30% of the SCD-associated SNPs, or 0% to 20% of the SCD-associated SNPs. In some cases, 2% to 95% of the SCD-associated SNPs in the engrafted population are corrected after a period of time after said administering such as, e.g., 2% to 80% of the SCD-associated SNPs, 2% to 70% of the SCD-associated SNPs, 2% to 60% of the SCD-associated SNPs, 2% to 50% of the SCD-associated SNPs, 2% to 40% of the SCD-associated SNPs, 2% to 30% of the SCD-associated SNPs, or 2% to 20% of the SCD-associated SNPs. The corrected SCD-associated SNPs in the engrafted population may remain corrected for a period of time after engraftment up to the lifespan of the individual. In some instances, 20% of the SCD-associated SNPs are corrected after a period of time ranging from 1 day to 6 months after administration, from 6 months to 12 months after administration, from 1 year to 2 years after administration, or for a period of time after administration that lasts up to the years in the individual's lifespan. In some cases, the period of time may be at least one month following said administering, at least 6 months following said administering, at least 1 year following said administering, or at least 2 years following said administering. In some cases, 2% to 95% of the SCD-associated SNPs remain corrected after a period of time such as, e.g., 2% to 90% of the SCD-associated SNPs, 2% to 80% of the SCD-associated SNPs, 2% to 70% of the SCD-associated SNPs, 2% to 60% of the SCD-associated SNPs, or 2% to 50% of the SCD-associated SNPs. In some instances, 20% of the SCD-associated SNPs remain corrected after a period of time ranging 1 day to 6 months after administration, from 6 months to 12 months after administration, from 1 year to 2 years after administration, or for a period of time after administration that lasts up to the years in the individual's lifespan. In some cases, the period of time is at least one month following said administering, at least 6 months following said administering, at least 1 year following said administering, or at least 2 years following said administering. In some cases, 2% to 95% of the total SCD alleles are the corrected SCD allele in the engrafted population of the treated individual after a period of time such as, e.g., 2% to 90% of the total SCD alleles, 2% to 80% of the total SCD alleles, 2% to 70% of the total SCD alleles, 2% to 60% of the total SCD alleles, or 2% to 50% of the total SCD alleles. The corrected SCD allele may remain corrected for a period of time ranging from 1 day to 6 months after administration, from 6 months to 12 months after administration, from 1 year to 2 years after administration, or for a period of time after administration that lasts up to the years in the individual's lifespan. In some cases, the period of time is at least one month following said administering, at least 6 months following said administering, at least 1 year following said administering, or at least 2 years following said administering.

In some cases, the engrafted population includes a population of HSCs having at least one corrected SCD-associated SNP that remains corrected for a period of time after administering the in vitro mixed population to the individual. The period of time may be at least one month following said administering, at least 6 months following said administering, at least 1 year following said administering, or at least 2 years following said administering. In some cases, the at least one corrected SCD-associated SNP remains corrected after said administering for the individual's lifetime. In some cases, 2% to 20% of HSCs in the engrafted population comprise at least one corrected SCD-associated SNP that remains corrected for a period of time; for example, 2% to 25% of HSCs, 2% to 30% of HSCs, 2% to 35% of HSCs, 2% to 40% of HSCs, 2% to 45% of HSCs, 2% to 50%, or 50% or more of HSCs in the engrafted population comprise at least one corrected SCD-associated SNP that remains corrected for a period of time after said administering. The population of HSCs having at least one corrected SCD-associated SNP that remains corrected for a period of time after said administering may provide for circulating red blood cells (RBCs) that comprise HbA. For example, the population of HSCs having at least one corrected SCD-associated SNP that remains corrected for a period of time after said administering may provide for circulating RBCs in the individual that comprise HbA, wherein at least 40% of the total circulating RBCs in the individual comprise HbA, at least 50% of the total circulating RBCs comprise HbA, at least 60% of the total circulating RBCs comprise HbA, at least 70% of the total circulating RBCs comprise HbA, at least 80% of the total circulating RBCs comprise HbA, or at least 90% of the total circulating RBCs comprise HbA. In some cases, 2% to 20% of HSCs having at least one corrected SCD-associated SNP that remains corrected for a period of time after said administering may provide for circulating RBCs that comprise HbA, where at least 40% of the total circulating RBCs in the individual comprise HbA.

In some cases, the method provides for circulating red blood cells (RBCs) in the individual that include zero, one, or two corrected SCD-associated SNPs after a period of time. The period of time may range from 1 day to 6 months after administration, from 6 months to 12 months after administration, from 1 year to 2 years after administration, or for a period of time after administration that lasts up to the years in the individual's lifespan. In some cases, the period of time is at least one month following said administering, at least 6 months following said administering, at least 1 year following said administering, or at least 2 years following said administering. In certain embodiments, 2% to 95% of circulating red blood cells in the individual comprise two non-corrected SCD-associated SNPs after a period of time such as, e.g., 2% to 90% of RBCs, 2% to 80% of RBCs, 2% to 70% of RBCs, 2% to 60% of RBCs, 2% to 50% of RBCs or 2% to 40% of RBCs. In certain embodiments, 2% to 95% of circulating red blood cells in the individual comprise only one corrected SCD-associated SNP after a period of time such as, e.g., 2% to 90% of RBCs, 2% to 80% of RBCs, 2% to 70% of RBCs, 2% to 60% of RBCs, 2% to 50%, or 2 to 40% of RBCs. In certain embodiments, 2% to 95% of circulating red blood cells in the individual comprise two corrected SCD-associated SNPs after a period of time such as, e.g., 2% to 90% of RBCs, 2% to 80% of RBCs, 2% to 70% of RBCs, 2% to 60% of RBCs, 2% to 50% of RBCs, or 2% to 40% of RBCs. In certain embodiments, 2% to 95% of circulating red blood cells in the individual comprise HbA after a period of time such as, e.g., 2% to 90% of RBCs, 2% to 80% of RBCs, 2% to 70% of RBCs, 2% to 60% of RBCs, 2% to 50% of RBCs, or 2% to 40% of RBCs. In some cases, at least 99% of circulating red blood cells in the individual comprise HbA after a period of time after administering the in vitro mixed population; for example, at least 95% of circulating RBCs, at least 90% of circulating RBCs, at least 85% of circulating RBCs, at least 80% of circulating RBCs, at least 75% of circulating RBCs, at least 70% of circulating RBCs, at least 65% of circulating RBCs, at least 60% of circulating RBCs, at least 55% of circulating RBCs, at least 50% of circulating RBCs, at least 45% of circulating RBCs, or at least 40% of circulating RBCs in the individual comprise HbA after a period of time after administering the in vitro mixed population. In some cases, 2% to 20% of the total SCD alleles are the corrected SCD allele in the population of circulating RBCs of the treated individual after a period of time.

The subject methods may provide an increase in circulating normal RBCs after a period of time. In some cases, the circulating RBCs have a wild type morphology after a period of time. By “wild type morphology” is meant a healthy RBC morphology, e.g., the morphology of a normal, mature RBC. Cells having a wild type morphology may have a normal size and may be, e.g., biconcave, disc-shaped, anuclear cells measuring approximately 7-8 microns in diameter with an internal volume of 80-100 fL. In certain embodiments, 2% to 95% of circulating RBCs have a wild type morphology after a period of time such as, e.g., 5% to 90% of RBCs, 10% to 80% of RBCs, 20% to 70% of RBCs, 30% to 60% of RBCs, or 40% to 50% of RBCs. The period of time may range from 1 day to 6 months after administration of the in vitro mixed population, from 6 months to 12 months after administration, from 1 year to 2 years after administration, or for a period of time after administration that lasts up to the years in the individual's lifespan. In some cases, the period of time is at least one month following said administering, at least 6 months following said administering, at least 1 year following said administering, or at least 2 years following said administering. In some cases, the circulating RBCs have improved survival relative to the survival of RBCs in untreated individuals. The circulating RBCs in a treated individual may survive for 70 days to 130 days such as, e.g., for 80 days to 120 days, for 90 days to 120 days, or for 100 days to 120 days. The circulating RBCs in a treated individual may survive for 2 to 100 more days such as, e.g., for 5 to 50 more days, for 10 to 30 more days, or for 15 to 20 more days than circulating RBCs in an untreated individual.

The administering may provide for the production of normal hemoglobin, e.g., as measured by HPLC. In some cases, the administering provides for production of hemoglobin A (HbA) in the individual. The administering may provide for a ratio of HbA to hemoglobin S (HbS) in the individual of at least 0.1:1.0, such as, e.g., at least 0.25:1.0, at least 0.5:1.0, at least 0.75:1.0, at least 1.0:1.0, at least 1.25:1.0, at least 1.5:1.0, or at least 1.75:1.0. In some cases, the administering provides for production of hemoglobin F (HbF) in the individual. In some cases, the administering provides for a ratio of HbF to HbS in the individual of at least 0.01:1.0 such as, e.g., at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0. In some cases, the administering provides for an amount of HbS that is less than 95% of the total hemoglobin in serum such as, e.g., less than 90%, less than 80%, less than 70%, less than 60%, less than 50%, less than 40%, less than 30%, less than 20%, less than 10%, or less than 5% of the total hemoglobin in serum. In some cases, the administering provides for an amount of HbA and HbF, e.g., the sum of the amount of HbA and the amount of HbF, that is 2% to 95% of the total hemoglobin in serum such as, e.g., 5% to 90% of the total hemoglobin in serum, 10% to 80% of the total hemoglobin in serum, 20% to 70% of the total hemoglobin in serum, 30% to 60% of the total hemoglobin in serum, or 40% to 50% of the total hemoglobin in serum. In some cases, the administering provides for an amount of HbA and HbF that is 50% of the total hemoglobin in serum or greater than 50% of the total hemoglobin in serum. In some cases, the administering provides for 2% to 95% HbS in serum such as, e.g., 5% to 90% HbS in serum, 10% to 80% HbS in serum, 20% to 70% HbS in serum, 30% to 60% HbS in serum, or 40% to 50% HbS in serum. In some cases, the administering provides 2% to 95% HbA in serum such as, e.g., 2% to 95% HbA in serum such as, e.g., 5% to 90% HbA in serum, 10% to 80% HbA in serum, 20% to 70% HbA in serum, 30% to 60% HbA in serum, or 30% to 50% HbA in serum. In some cases, the administering provides for 2% to 45% HbF in serum such as, e.g., for 10% to 45% HbF in serum, for 15% to 45% HbF in serum, 20% to 45% HbF in serum, or for 25% to 40% HbF in serum. The production, e.g., amounts, of HbA, HbF, and/or HbS may be determined after a period of time. The ratios of HbA:HbS and/or HbA:HbF may be determined after a period of time. The period of time may range from 1 day to 6 months after administration, from 6 months to 12 months after administration, from 1 year to 2 years after administration, or for a period of time after administration that lasts up to the years in the individual's lifespan. In some cases, the period of time is at least one month following said administering, at least 6 months following said administering, at least 1 year following said administering, or at least 2 years following said administering.

The methods of treating may provide the reduction of adverse symptoms associated with sickle cell disease (SCD) after a period of time after administering the in vitro mixed population. The period of time may range from 1 day to 6 months after administration, from 6 months to 12 months after administration, from 1 year to 2 years after administration, or for a period of time after administration that lasts up to the years in the individual's lifespan. In some cases, the period of time is at least one month following said administering, at least 6 months following said administering, at least 1 year following said administering, or at least 2 years following said administering. In some cases, the methods result in the reduction of the clinical presentation of SCD. In some cases, the methods result in the reduction in the frequency of the clinical presentation of SCD. In some cases, the methods result in the reduction in the severity of the clinical presentation of SCD. The methods may result in the elimination or prevention of the clinical presentation of SCD. The clinical presentation of SCD may include, e.g., pain crises requiring hospitalization, organ damage, kidney damage, pulmonary events (e.g., stroke), spleen damage, and anemia. In some cases, the methods result in the reduction in the frequency of pain crises requiring hospitalization in a treated individual by 2% to 95% compared to the frequency in the individual before treatment or in an untreated individual such as, e.g., by 5% to 90%, by 10% to 80%, by 20% to 70%, by 30% to 60%, or by 40% to 50%. In some cases, the methods result in the reduction in the severity of pain crises requiring hospitalization in a treated individual by 2% to 95% compared to the severity in the individual before treatment or in an untreated individual such as, e.g., by 5% to 90%, by 10% to 80%, by 20% to 70%, by 30% to 60%, or by 40% to 50%. In some cases, the methods result in the reduction in frequency of organ damage in a treated individual by 2% to 95% compared to the frequency in the individual before treatment or in an untreated individual such as, e.g., by 5% to 90%, by 10% to 80%, by 20% to 70%, by 30% to 60%, or by 40% to 50%. In some cases, the methods result in the reduction in the severity of organ damage in a treated individual by 2% to 95% compared to the severity in the individual before treatment or in an untreated individual such as, e.g., by 5% to 90%, by 10% to 80%, by 20% to 70%, by 30% to 60%, or by 40% to 50%. In some cases, the methods result in the reduction in the frequency of kidney damage in a treated individual by 2% to 95% compared to the frequency in the individual before treatment or in an untreated individual such as, e.g., by 5% to 90%, by 10% to 80%, by 20% to 70%, by 30% to 60%, or by 40% to 50%. In some cases, the methods result in the reduction in the severity of kidney damage in a treated individual by 2% to 95% compared to the severity in the individual before treatment or in an untreated individual such as, e.g., by 5% to 90%, by 10% to 80%, by 20% to 70%, by 30% to 60%, or by 40% to 50%. In some cases, the methods result in the reduction in the frequency of pulmonary events, e.g., stroke, in a treated individual by 2% to 95% compared to the frequency in the individual before treatment or in an untreated individual such as, e.g., by 5% to 90%, by 10% to 80%, by 20% to 70%, by 30% to 60%, or by 40% to 50%. In some cases, the methods result in the reduction in the severity of pulmonary events, e.g., stroke, in a treated individual by 2% to 95% compared to the severity in the individual before treatment or in an untreated individual such as, e.g., by 5% to 90%, by 10% to 80%, by 20% to 70%, by 30% to 60%, or by 40% to 50%. In some cases, the methods result in the reduction in frequency of symptoms of anemia in a treated individual by 2% to 95% compared to the frequency in the individual before treatment or in an untreated individual such as, e.g., by 5% to 90%, by 10% to 80%, by 20% to 70%, by 30% to 60%, or by 40% to 50%. In some cases, the methods result in the reduction in severity of symptoms of anemia in a treated individual by 2% to 95% compared to the severity in the individual before treatment or in an untreated individual such as, e.g., by 5% to 90%, by 10% to 80%, by 20% to 70%, by 30% to 60%, or by 40% to 50%. In some cases, the methods result in the reduction in the frequency of splenic complications or damage to the spleen, e.g., splenic sequestration, in a treated individual by 2% to 95% compared to the frequency in the individual before treatment or in an untreated individual such as, e.g., by 5% to 90%, by 10% to 80%, by 20% to 70%, by 30% to 60%, or by 40% to 50%. In some cases, the methods result in the reduction in the severity of splenic complications or damage to the spleen, e.g., splenic sequestration, in a treated individual by 2% to 95% compared to the severity in the individual before treatment or in an untreated individual such as, e.g., by 5% to 90%, by 10% to 80%, by 20% to 70%, by 30% to 60%, or by 40% to 50%. In some cases, the methods result in the reduction in hydroxyurea use by a treated individual by 2% to 95% compared to the hydroxyurea use in the individual before treatment or in an untreated individual such as, e.g., by 5% to 90%, by 10% to 80%, by 20% to 70%, by 30% to 60%, or by 40% to 50%. In some cases, the methods result in the reduction in the number of RBC transfusions to a treated individual by 2% to 95% compared to the number of transfusions to the individual before treatment or to an untreated individual such as, e.g., by 5% to 90%, by 10% to 80%, by 20% to 70%, by 30% to 60%, or by 40% to 50%. In some cases, the methods result in the increase in chance for survival of a treated individual by 2% to 95% compared to the chance for survival of the individual before treatment or of an untreated individual such as, e.g., by 5% to 90%, by 10% to 80%, by 20% to 70%, by 30% to 60%, or by 40% to 50%. In some cases, the methods result in the increase in years of survival of a treated individual by 1 year to 50 years compared to the years of survival for the individual before treatment or for an untreated individual such as, e.g., by 5 years to 40 years, by 10 years to 30 years or by 15 years to 20 years.

### Kits

Aspects of the present disclosure include a kit for treating sickle cell disease (SCD) in an individual. The kit may include A) a stem cell mobilization agent that provides for mobilization of hematopoietic stem cells; and B) a genome-editing composition comprising: a) a ribonucleoprotein (RNP) complex comprising: i) a class 2 CRISPR/Cas effector polypeptide, or a nucleic acid comprising a nucleotide sequence encoding the class 2 CRISPR/Cas effector polypeptide; and ii) a guide RNA; and b) a donor DNA template comprising a nucleotide sequence that provides for correction of an SCD-associated single nucleotide polymorphism in a globin gene.

Where desired, the kits may further include one or more additional components that find use in an application, e.g., reagents, buffers, etc. Any or all of the kit components may be present in sterile packaging, as desired. In some cases, one or more kit components may be present in a container, e.g., a sterile container, such as a syringe. In some cases, the stem cell mobilization agent is plerixafor. In some cases, the class 2 CRISPR/Cas effector polypeptide is a type II CRIPSR/Cas effector polypeptide, as described above. The guide RNA may include any suitable guide RNA, as described above. The donor DNA template can include any suitable donor DNA template, as described above.

In addition to the above-mentioned components, a subject kit may further include instructions for using the components of the kit, e.g., to practice the subject methods. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., a portable flash drive, CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-56 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of modifying a globin gene in the genome of a hematopoietic stem/progenitor cell (HSPC), the method comprising:

A) obtaining HSPCs from an individual having a globin gene comprising a sickle cell disease (SCD)-associated single-nucleotide polymorphism (SNP), wherein said obtaining comprises:
a) administering to the individual an amount of a stem cell mobilization agent effective to mobilize CD34⁺ HSPCs; and
b) collecting the mobilized CD34⁺ HSPCs from the individual, thereby generating an in vitro population of CD34⁺ HSPCs;

B) contacting the in vitro population of CD34⁺ HSPCs with a genome editing composition comprising:
a) a ribonucleoprotein (RNP) complex comprising:
   i) a class 2 CRISPR/Cas effector polypeptide, or a nucleic acid comprising a nucleotide sequence encoding the class 2 CRISPR/Cas effector polypeptide; and
   ii) a guide RNA; and
b) a donor DNA template comprising a nucleotide sequence that provides for correction of the SCD-associated SNP in the globin gene,

thereby generating an in vitro mixed population, wherein at least 2% of the SCD-associated SNPs are corrected in the in vitro mixed population.

Aspect 2. The method of aspect 1, wherein the class 2 CRISPR/Cas effector polypeptide is a type II CRISPR/Cas effector polypeptide.

Aspect 3. The method of aspect 2, wherein the class 2 CRISPR/Cas effector polypeptide is a Cas9 protein and the corresponding CRISPR/Cas guide RNA is a Cas9 guide RNA.

Aspect 4. The method of aspect 1, wherein the class 2 CRISPR/Cas effector polypeptide is a type V or type VI CRISPR/Cas effector polypeptide.

Aspect 5. The method of aspect 4, wherein the class 2 CRISPR/Cas effector polypeptide is a Cpf1 protein, a C2c1 protein, a C2c3 protein, or a C2c2 protein.

Aspect 6. The method of aspect 4, wherein the class 2 CRISPR/Cas effector polypeptide is a Cas12 enzyme.

Aspect 7. The method of aspect 4, wherein the class 2 CRISPR/Cas effector polypeptide is a Cas13 enzyme.

Aspect 8. The method of aspect 1, wherein the class 2 CRISPR/Cas effector polypeptide is a high-fidelity variant.

Aspect 9. The method of any one of aspects 1-8, wherein the guide RNA comprises one or more nucleic acid modifications.

Aspect 10. The method of aspect 9, wherein the first three nucleotides at the 5′ end of the guide RNA comprise nucleic acid modifications.

Aspect 11. The method of aspect 10, wherein the nucleic acid modifications comprise one or more of a modified nucleobase, a modified backbone or non-natural internucleoside linkage, a modified sugar moiety, a Locked Nucleic Acid, and a Peptide Nucleic acid.

Aspect 12. The method of any one of aspects 1-11, wherein the stem cell mobilization agent is plerixafor.

Aspect 13. The method of any one of aspects 1-12, wherein the SCD-associated SNP is an A-to-T substitution at position 170 of the nucleotide sequence depicted in FIG. 15.

Aspect 14. The method of any one of aspects 1-13, wherein the donor DNA template comprises the nucleotide sequence

(SEQ ID NO: 1126)
5′-tcagggcagagccatctattgcttacaTTTGCTTCTGACACAACTGTG
TTCACTAGCAACCTCAAACAGACACCATGGTGCACCTGACTCCTgaaGAGA
AGTCTGCGGTTACTGCCCTGTGGGGCAAGGTGAACGTGGATGAAGTTGGTG
GTGAGGCCCTGGGCAGGT-3′.

Aspect 15. The method of any one of aspects 1-14, wherein the guide RNA targeting segment comprises the nucleotide sequence 5′-CUUGCCCCACAGGGCAGUAA-3′ (SEQ ID NO: 1128).

Aspect 16. The method of any one of aspects 1-15, wherein 2% to 50% of the SCD-associated SNPs in the in vitro mixed population have been corrected.

Aspect 17. The method of aspect 16, wherein 35% of the SCD-associated SNPs in the in vitro mixed population have been corrected.

Aspect 18. The method of any one of aspects 1-17, wherein from 2% to 25% of the SCD-associated SNPs in the in vitro mixed population have been corrected.

Aspect 19. The method of any one of aspects 1-18, wherein from 2% to 20% of cells of the in vitro mixed population comprise only one corrected SCD-associated SNP.

Aspect 20. The method of any one of aspects 1-18, wherein from 2% to 20% of cells of the in vitro mixed population comprise two corrected SCD-associated SNPs.

Aspect 21. A method of treating sickle cell disease (SCD) in an individual, the method comprising:

a) modifying a globin gene in the genome of a hematopoietic stem/progenitor cell (HSPC) obtained from the individual according to the method of any one of aspects 1-20, thereby generating an in vitro mixed population, wherein at least 2% of the SCD-associated SNPs are corrected in the in vitro mixed population; and

b) administering the in vitro mixed population to the individual, thereby treating the SCD in the individual.

Aspect 22. The method of aspect 21, wherein administering the mixed population produces an engrafted population comprising hematopoietic stem cells (HSCs).

Aspect 23. The method of aspect 22, wherein from 2% to 20% of HSCs in the engrafted population comprise at least one corrected SCD-associated SNP for a period of time of at least one month following said administering.

Aspect 24. The method of aspect 22, wherein from 2% to 20% of cells of the engrafted population comprise only one corrected SCD-associated SNP for a period of time of at least one month following said administering.

Aspect 25. The method of aspect 22, wherein 2% to 20% of cells of the engrafted population retain two corrected SCD-associated SNPs for a period of time of at least one month following said administering.

Aspect 26. The method of any one of aspects 22-25, wherein 2% to 50% of the SCD-associated SNPs are corrected for a period of time of at least one month following said administering.

Aspect 27. The method of aspect 26, wherein 20% of the SCD-associated SNPs are corrected for a period of time of at least one month following said administering.

Aspect 28. The method of any one of aspects 22-27, wherein at least 40% of circulating red blood cells in the individual comprise HbA for a period of time of at least one month following said administering.

Aspect 29. The method of aspect 28, wherein 2% to 95% of circulating RBCs have a wild type morphology for a period of time of at least one month following said administering.

Aspect 30. The method of any one of aspects 21-29, wherein the method comprises administering 10⁶ to 10⁷ cells from the in vitro mixed population per kilogram of body weight of the individual.

Aspect 31. The method of any one of aspects 21-30, wherein the guide RNA comprises one or more nucleic acid modifications.

Aspect 32. The method of aspect 31, wherein the first three nucleotides at the 5′ end of the guide RNA comprise nucleic acid modifications.

Aspect 33. The method of aspect 32, wherein the nucleic acid modifications comprise one or more of a modified nucleobase, a modified backbone or non-natural internucleoside linkage, a modified sugar moiety, a Locked Nucleic Acid, and a Peptide Nucleic acid.

Aspect 34. The method of any one of aspects 21-33, wherein said administering provides for production of hemoglobin A (HbA) in the individual.

Aspect 35. The method of aspect 34, wherein said administering provides for a ratio of HbA to hemoglobin S (HbS) in the individual of at least 0.1:1.0.

Aspect 36. The method of aspect 34, wherein said administering provides for a ratio of HbA to HbS in the individual of at least 0.25:1.0.

Aspect 37. The method of any one of aspects 21-36, wherein said administering provides for production of hemoglobin F (HbF) in the individual.

Aspect 38. The method of any one of aspects 21-37, wherein said administering provides for an amount of HbS that is less than 50% of the total hemoglobin in serum.

Aspect 39. The method of aspect any one of aspects 21-38, wherein said administering provides for an amount of HbA and HbF that is at least 40% of the total hemoglobin in serum.

Aspect 40. The method of any one of aspects 21-39, wherein said administering provides for an amount of HbA and HbF that is at least 50% of the total hemoglobin in serum.

Aspect 41. The method of any one of aspects 21-40, wherein said administering provides for an amount of HbA and HbF that is at least 60% of the total hemoglobin in serum.

Aspect 42. A kit for treating sickle cell disease (SCD) in an individual, the kit comprising:

A) a stem cell mobilization agent that provides for mobilization of hematopoietic stem cells; and

B) a genome-editing composition comprising:
a) a ribonucleoprotein (RNP) complex comprising:
   i) a class 2 CRISPR/Cas effector polypeptide, or a nucleic acid comprising a nucleotide sequence encoding the class 2 CRISPR/Cas effector polypeptide; and
   ii) a guide RNA; and
b) a donor DNA template comprising a nucleotide sequence that provides for correction of an SCD-associated single nucleotide polymorphism in a globin gene.

Aspect 43. The kit of aspect 42, wherein the stem cell mobilization agent is plerixafor.

Aspect 44. The kit of aspect 42 or 43, wherein the class 2 CRISPR/Cas effector polypeptide is a type II CRISPR/Cas effector polypeptide.

Aspect 45. The kit of aspect 44, wherein the class 2 CRISPR/Cas effector polypeptide is a Cas9 protein and the corresponding CRISPR/Cas guide RNA is a Cas9 guide RNA.

Aspect 46. The kit of aspect 42 or 43, wherein the class 2 CRISPR/Cas effector polypeptide is a type V or type VI CRISPR/Cas effector polypeptide.

Aspect 47. The kit of aspect 46, wherein the class 2 CRISPR/Cas effector polypeptide is a Cpf1 protein, a C2c1 protein, a C2c3 protein, or a C2c2 protein.

Aspect 48. The kit of aspect 46, wherein the class 2 CRISPR/Cas effector polypeptide is a Cas12 enzyme.

Aspect 49. The kit of aspect 46, wherein the class 2 CRISPR/Cas effector polypeptide is a Cas13 enzyme.

Aspect 50. The kit of aspect 42, wherein the class 2 CRISPR/Cas effector polypeptide is a high-fidelity variant.

Aspect 51. The kit of any one of aspects 42-50, wherein the guide RNA comprises one or more nucleic acid modifications.

Aspect 52. The kit of aspect 51, wherein the first three nucleotides at the 5′ end of the guide RNA comprise nucleic acid modifications.

Aspect 53. The kit of aspect 52, wherein the nucleic acid modifications comprise one or more of a modified nucleobase, a modified backbone or non-natural internucleoside linkage, a modified sugar moiety, a Locked Nucleic Acid, and a Peptide Nucleic acid.

Aspect 54. The kit of any one of aspects 42-53, wherein the SCD-associated SNP is an A-to-T substitution at position 170 of the nucleotide sequence depicted in FIG. 15.

Aspect 55. The kit of any one of aspects 42-54, wherein the donor DNA template comprises the nucleotide sequence

(SEQ ID NO: 1126)
5′-tcagggcagagccatctattgcttacaTTTGCTTCTGACACAACTGTG
TTCACTAGCAACCTCAAACAGACACCATGGTGCACCTGACTCCTgaaGAGA
AGTCTGCGGTTACTGCCCTGTGGGGCAAGGTGAACGTGGATGAAGTTGGTG
GTGAGGCCCTGGGCAGGT-3′.

Aspect 56. The kit of any one of aspects 42-55, wherein the guide RNA targeting segment comprises the nucleotide sequence 5′-CUUGCCCCACAGGGCAGUAA-3′ (SEQ ID NO: 1128).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

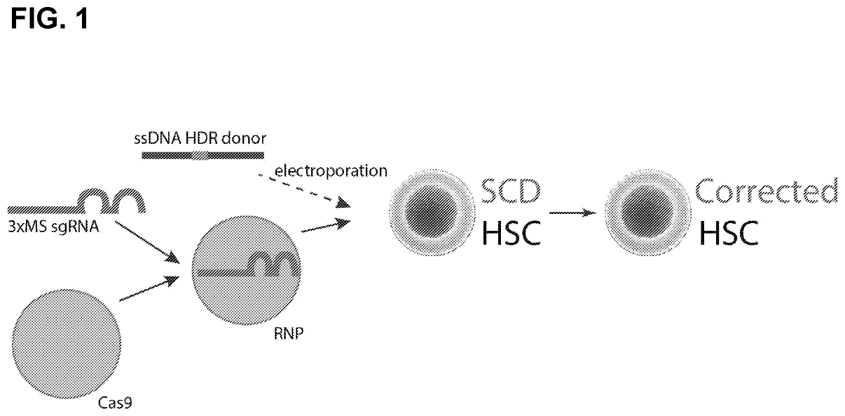

FIG. 1 shows the use of Cas9 ribonucleoprotein and short ssDNA HDR donor to genetically edit human cells. Cas9 RNP was assembled by mixing in vitro, along with a ssDNA donor with the desired mutation, and delivered to human cells by electroporation. In the example provided, this process can convert a blue fluorescent protein (BFP)-encoding gene to a green fluorescent protein (GFP)-encoding gene with 25% efficiency with no selection.

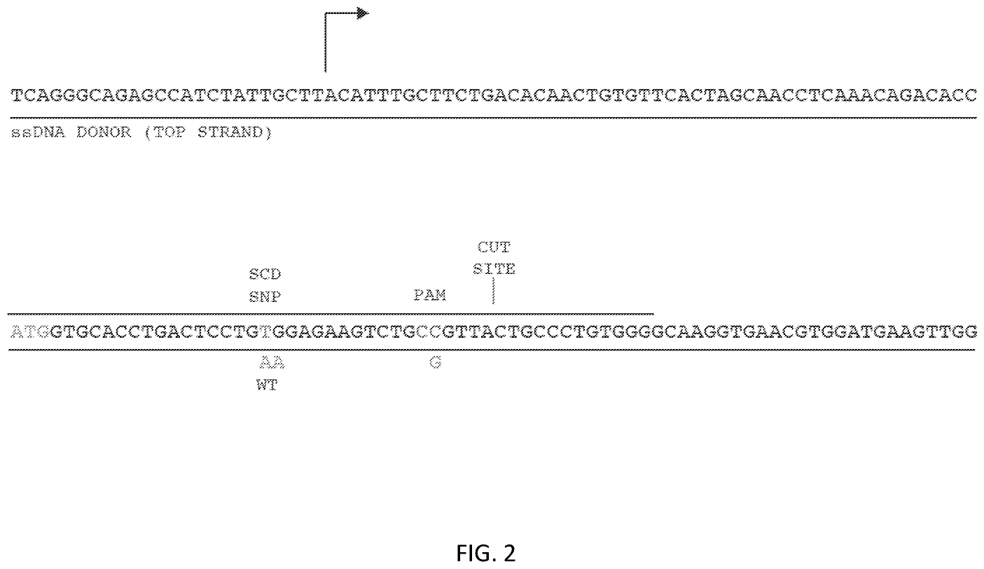

FIG. 2 shows an outline of a method of targeting the sickle mutation in CD34⁺ HSPCs. The targeted region of the adult ß-globin gene with the sickle SNP is shown. The gene is targeted with a guide, G10 which binds to the indicated region, making a double-strand break at the cut site indicated. The ssDNA HDR donor CJ6A, which matches the sense strand (indicated) is incorporated into the cut genomic DNA, introducing a corrected “wild-type” sequence, along with mutations that prevent binding of G10 to the target gene, resulting in gene correction from SCD to wild-type.

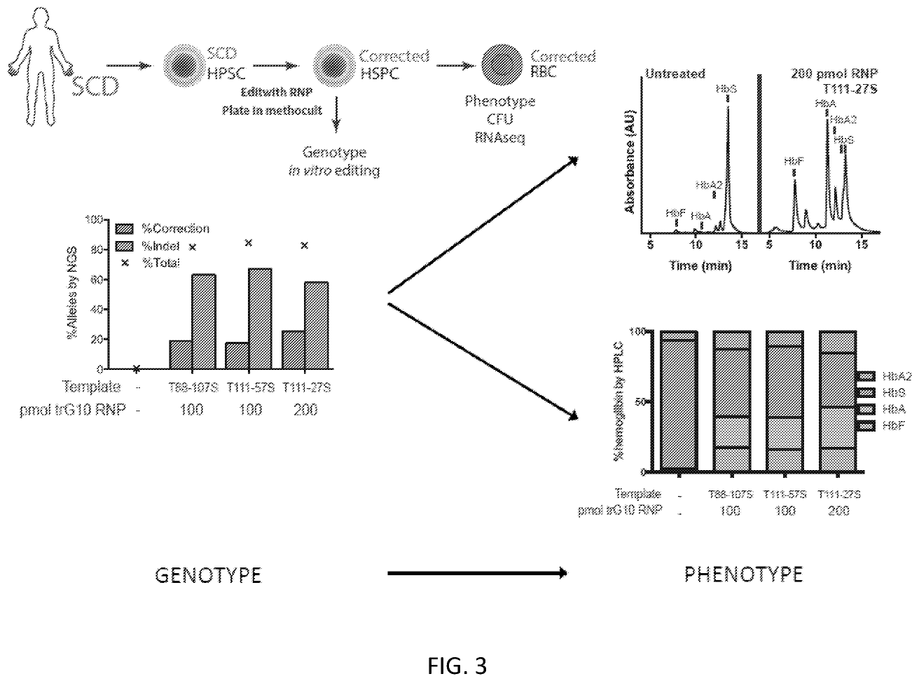

FIG. 3 illustrates consequences of gene correction on CD34⁺ HSPCs. Three samples were electroporated into CD34⁺ HSPCs with the reagents described above, leading to 15-20% correction. When this mixture of corrected cells was differentiated into red blood cells, they expressed reduced sickle hemoglobin (HbS), and increased wild-type adult hemoglobin (HbA) and fetal hemoglobin (HbF).

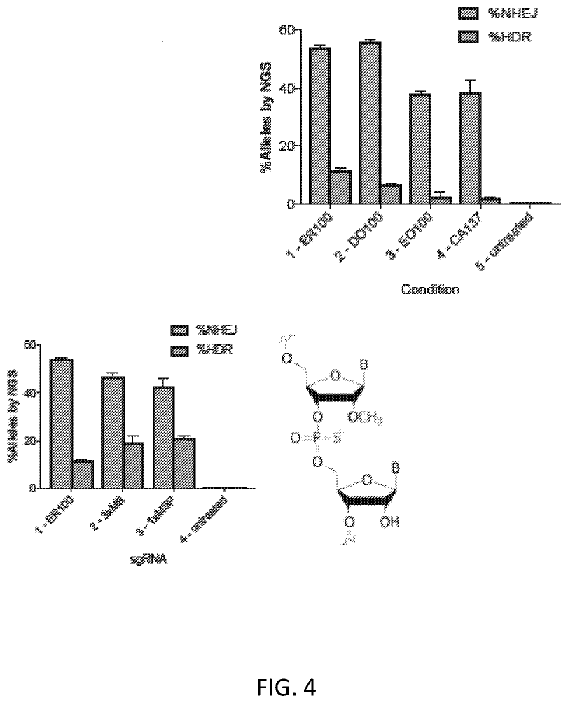

FIG. 4 shows optimization of reagents and techniques. Different electroporation codes led to different levels of HDR at the SCD SNP. ER100 code on the Lonza 4d electroporator was used. Synthetic RNAs worked well for correction, and the “3×MS” protection (indicated) was also used.

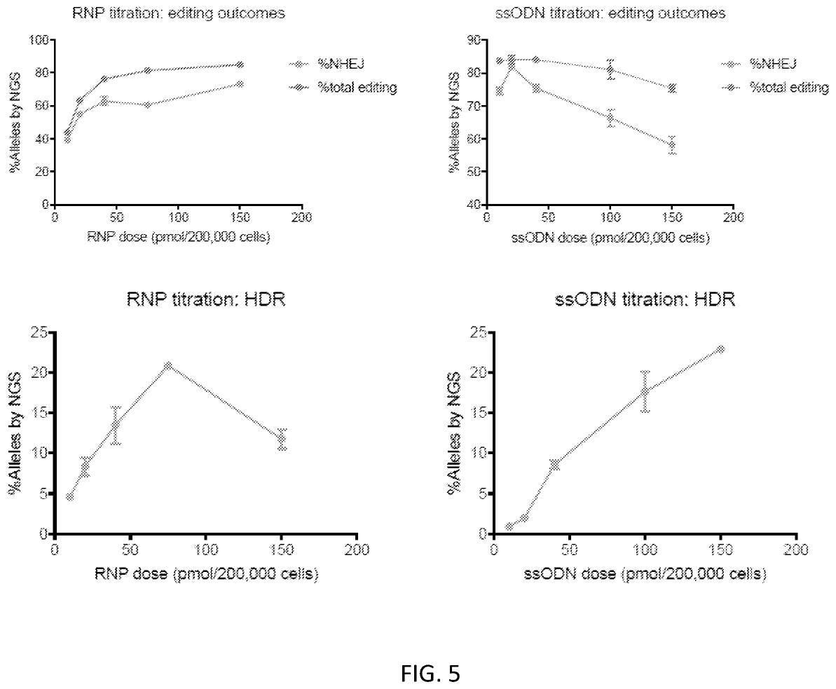

FIG. 5 shows the determination of the optimal dose of the RNP (with the 3×MS-protected G10 guide) and ssDNA donor (ssODN). The RNP was optimal in terms of HDR at 75 pmol per 100,000 cells (in a 20 μL electroporation volume), and the ssODN was optimal at 100 pmol.

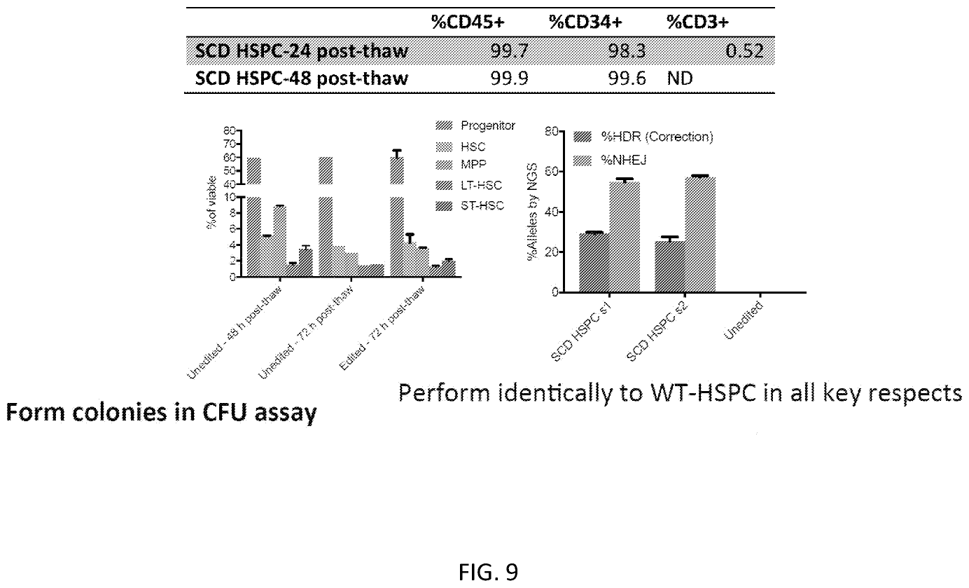

FIG. 9 shows properties of CD34⁺ HSPC collected from Plerixafor mobilization as described above. These cells were almost entirely CD34⁺, and there was an absence of undesired T cells. Analysis by flow cytometry indicated the presence of long-term engrafting stem cells (LT-HSC) in the mixture. Correction of these cells was robust, with 35% correction in two in vitro tests, better than for other sources of cells. These cells formed colonies in soft agar with cytokines (CFU assay), indicating healthy cells after correction.

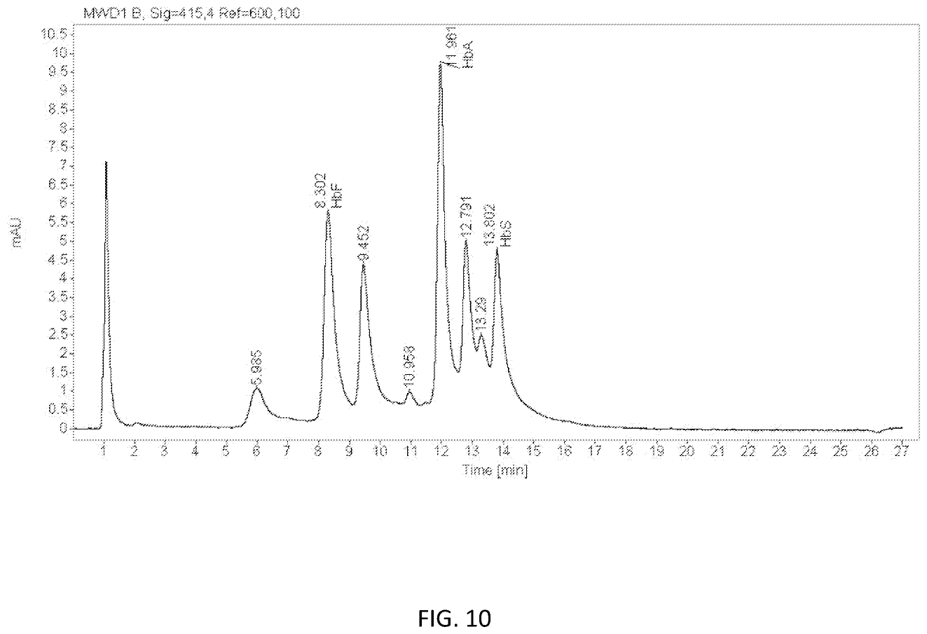

FIG. 10 shows HPLC trace of corrected HSPC mobilized with Plerixafor after differentiation into erythrocytes. These cells expressed 77% non-sickle hemoglobin (compared to <5% for untreated cells) by this technique.

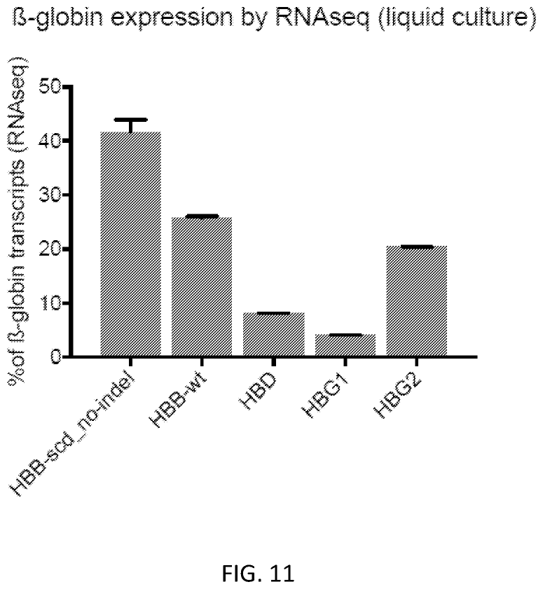

FIG. 11 shows RNAseq analysis of globin mRNA expression of the same cells as in FIG. 8. These cells expressed 55% non-sickle globin.

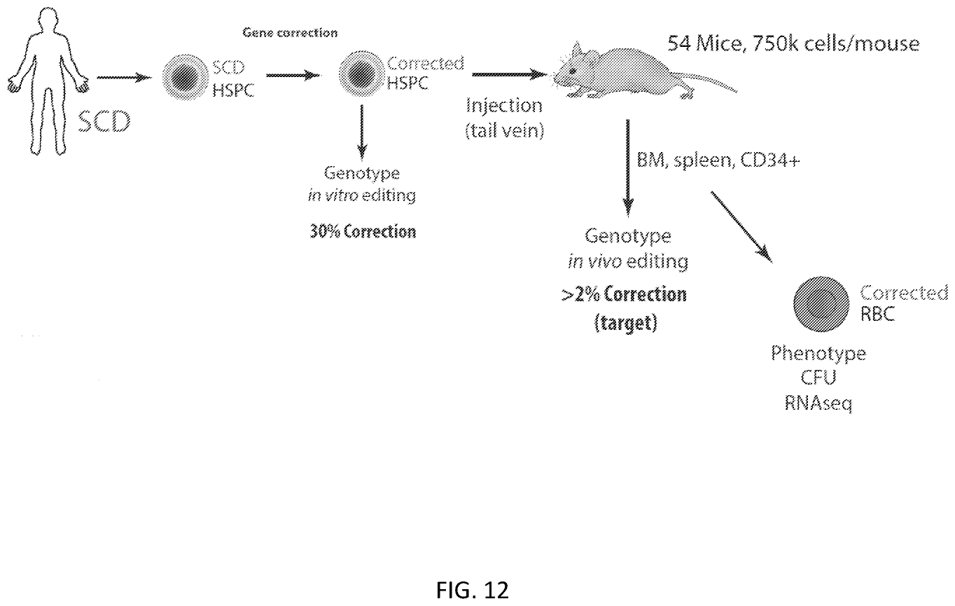

FIG. 12 provides a schematic illustrating an assay for determining the long-term engraftment potential of the corrected HSPC from Plerixafor mobilization of sickle cell disease patient. HSPC are corrected using the protocol optimized above in large batches. Corrected cell mixtures are cultured for one day and then a sample is removed to estimate correction by next-generation sequencing. The remaining cells are injected into NBSGW mice by tail vein injection (500,000 to 1 million cells per mouse). Mice are kept in cages for 4 months as corrected cells engraft in the bone marrow. After sacrifice, correction of human cells in the bone marrow is estimated by next-generation sequencing. For some mice, CD34⁺ cells are sorted out, and analyzed for engraftment and differentiation potential by the CFU assay, and differentiation into erythrocytes with characterization by HPLC and RNAseq.

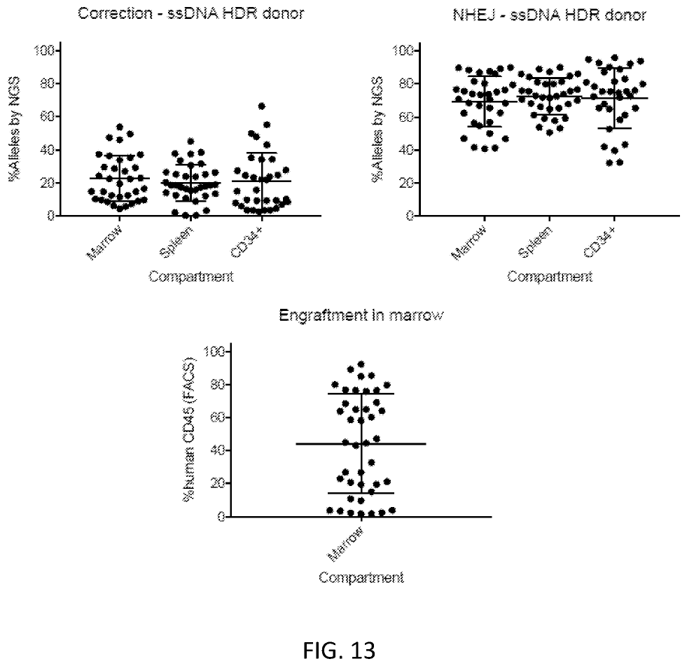

FIG. 13 provides characteristics of corrected HSCs after engraftment for 4 months in the NBSGW mice. Correction averaged 22% in the bone marrow, 24% in the spleen, and 20% in the CD34⁺ cells sorted from the marrow by FACS. NHEJ was high in all compartments, >65% (meaning that the sickle allele was greatly reduced to about 15%). Engraftment of human cells in the bone marrow averaged 45%.

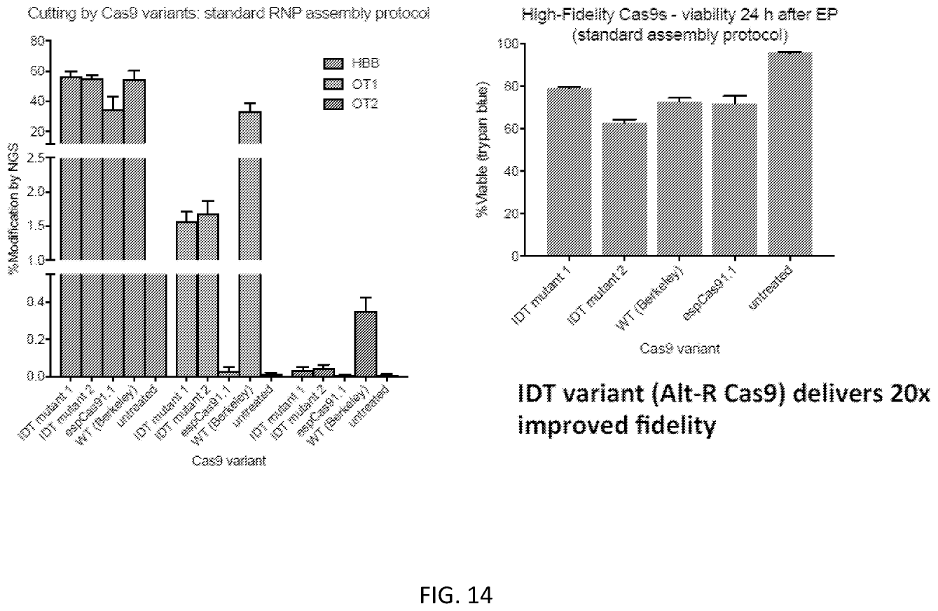

FIG. 14 shows reduction of off-target activity using IDT HiFi Cas9 mutant #1. This variant of Cas9 had mutation at the on-target site equal to wild-type Cas9, and had greatly reduced mutation at off-target sites OT1 and OT2, compared to wild-type Cas9. Other variants (espCas9 1.1) exhibited poor mutation at the on-target site. Viability of the edited HSPC after treatment with the HiFi Cas9 mutant 1 was high, ˜80%.

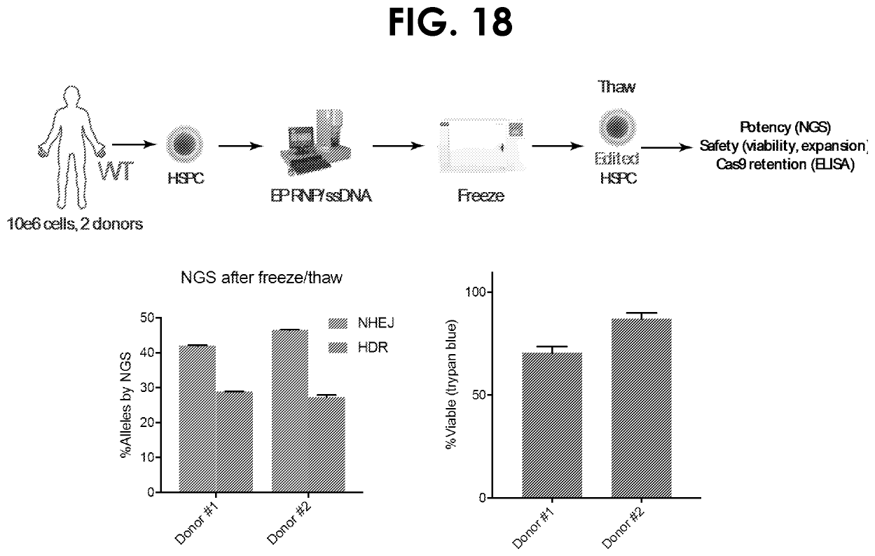

FIG. 18 depicts electroporation of harvested cells, and storage and use of the electroporated cells. 10 million cells from 2 different healthy people were electroporated using a maxcyte instrument with: 3.3 μM Cas9 protein, 3.96 μM G10-3×MS sgRNA in MaxCYte electroporation buffer at a volume of 100 μL, with power setting 7. Cells are cultured for 24 hours after electroporation before freezing in controlled-rate freezer. This is small-scale version of a protocol for clinical-scale production of gene-corrected cells. The frozen cells were thawed, cultured for 5 days and genotyped by next-generation sequencing (NGS), with HDR at the SCD SNP as indicated. Cells were viable and healthy, as indicated.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of modifying a globin gene in the genome of a hematopoietic stem/progenitor cell (HSPC), the method comprising:
A) obtaining HSPCs from an individual having a globin gene comprising a sickle cell disease (SCD)-associated single-nucleotide polymorphism (SNP), wherein said obtaining comprises:
a) administering to the individual an amount of a stem cell mobilization agent effective to mobilize CD34⁺ HSPCs; and
b) collecting the mobilized CD34⁺ HSPCs from the individual, thereby generating an in vitro population of CD34⁺ HSPCs;
B) contacting the in vitro population of CD34⁺ HSPCs with a genome editing composition comprising:
a) a ribonucleoprotein (RNP) complex comprising:
i) a class 2 CRISPR/Cas effector polypeptide, or a nucleic acid comprising a nucleotide sequence encoding the class 2 CRISPR/Cas effector polypeptide; and
ii) a guide RNA; and
b) a donor DNA template comprising a nucleotide sequence that provides for correction of the SCD-associated SNP in the globin gene,
thereby generating an in vitro mixed population, wherein at least 2% of the SCD-associated SNPs are corrected in the in vitro mixed population.

2. The method of claim 1, wherein the class 2 CRISPR/Cas effector polypeptide is a type II CRISPR/Cas effector polypeptide.

3. The method of claim 2, wherein the class 2 CRISPR/Cas effector polypeptide is a Cas9 protein and the corresponding CRISPR/Cas guide RNA is a Cas9 guide RNA.

4. The method of claim 1, wherein the class 2 CRISPR/Cas effector polypeptide is a type V or type VI CRISPR/Cas effector polypeptide.

5. The method of claim 4, wherein the class 2 CRISPR/Cas effector polypeptide is a Cpf1 protein, a C2c1 protein, a C2c3 protein, or a C2c2 protein.

6. The method of claim 4, wherein the class 2 CRISPR/Cas effector polypeptide is a Cas12 enzyme.

7. The method of claim 4, wherein the class 2 CRISPR/Cas effector polypeptide is a Cas13 enzyme.

8. The method of claim 1, wherein the class 2 CRISPR/Cas effector polypeptide is a high-fidelity variant.

9. The method of claim 1, wherein the guide RNA comprises one or more nucleic acid modifications.

10. The method of claim 9, wherein the first three nucleotides at the 5′ end of the guide RNA comprise nucleic acid modifications.

11. The method of claim 10, wherein the nucleic acid modifications comprise one or more of a modified nucleobase, a modified backbone or non-natural internucleoside linkage, a modified sugar moiety, a Locked Nucleic Acid, and a Peptide Nucleic acid.

12. The method of claim 1, wherein the stem cell mobilization agent is plerixafor.

13. The method of claim 1, wherein the SCD-associated SNP is an A-to-T substitution at position 170 of the nucleotide sequence depicted in FIG. 15.

14. The method of claim 1, wherein the donor DNA template comprises the nucleotide sequence
(SEQ ID NO: 1126)
5′-tcagggcagagccatctattgcttacaTTTGCTTCTGACACAACTGTG
TTCACTAGCAACCTCAAACAGACACCATGGTGCACCTGACTCCTgaaGAGA
AGTCTGCGGTTACTGCCCTGTGGGGCAAGGTGAACGTGGATGAAGTTGGTG
GTGAGGCCCTGGGCAGGT-3′.

15. The method of claim 1, wherein the guide RNA targeting segment comprises the nucleotide sequence 5′-CUUGCCCCACAGGGCAGUAA-3′ (SEQ ID NO: 1128).

16. The method of claim 1, wherein 2% to 50% of the SCD-associated SNPs in the in vitro mixed population have been corrected.

17. The method of claim 16, wherein 35% of the SCD-associated SNPs in the in vitro mixed population have been corrected.

18. The method of claim 1, wherein from 2% to 25% of the SCD-associated SNPs in the in vitro mixed population have been corrected.

19. The method of claim 1, wherein from 2% to 20% of cells of the in vitro mixed population comprise only one corrected SCD-associated SNP.

20. The method of claim 1, wherein from 2% to 20% of cells of the in vitro mixed population comprise two corrected SCD-associated SNPs.

21. A method of treating sickle cell disease (SCD) in an individual, the method comprising:
a) modifying a globin gene in the genome of a hematopoietic stem/progenitor cell (HSPC) obtained from the individual according to the method of claim 1, thereby generating an in vitro mixed population, wherein at least 2% of the SCD-associated SNPs are corrected in the in vitro mixed population; and
b) administering the in vitro mixed population to the individual, thereby treating the SCD in the individual.

22.-41. (canceled)

42. A kit for treating sickle cell disease (SCD) in an individual, the kit comprising:
A) a stem cell mobilization agent that provides for mobilization of hematopoietic stem cells; and
B) a genome-editing composition comprising:
a) a ribonucleoprotein (RNP) complex comprising:
i) a class 2 CRISPR/Cas effector polypeptide, or a nucleic acid comprising a nucleotide sequence encoding the class 2 CRISPR/Cas effector polypeptide; and
ii) a guide RNA; and
b) a donor DNA template comprising a nucleotide sequence that provides for correction of an SCD-associated single nucleotide polymorphism in a globin gene.

43.-56. (canceled)
